(19) United States
(12) Patent Application Publication
(10) Pub. No.: US 2024/0279684 A1
(43) Pub. Date: Aug. 22, 2024

(54) CYTOSOLIC DELIVERY OF GENOME EDITING TOOLS

(71) Applicant: SAPREME TECHNOLOGIES B.V., Bilthoven (NL)

(72) Inventors: Ruben POSTEL, Bilthoven (NL); Alexander WENG, Berlin (DE); Matthias Friedrich MELZIG, Berlin (DE); Simko SAMA, Berlin (DE)

(21) Appl. No.: 18/564,885

(22) PCT Filed: May 19, 2022
(86) PCT No.: PCT/NL2022/050271

§ 371 Date: Nov. 28, 2023

(30) Foreign Application Priority Data
May 28, 2021 (NL) 2028321

(51) Int. Cl. C12N 15/87; C12N 9/22; C12N 15/11; C12N 15/88; C12N 15/90

(52) U.S. Cl. CPC C12N 15/87; C12N 9/22; C12N 15/11; C12N 15/88; C12N 15/907; C12N 2310/20; C12N 2800/80

(57) ABSTRACT

A first aspect of the invention relates to the use of a saponin in the in vitro delivery of a nucleic acid into a cell. Typically, the nucleic acid is plasmid DNA, e.g. with a relatively large size of at least 5.5 kbp. In embodiments, the nucleic acid is transfected into cells in the presence of saponin GE1741 and/or saponin SO1861. A second aspect of the invention relates to a method for delivering a nucleic acid encoding for a CRISPR/Cas construct into a cell in vitro. Typically, the nucleic acid is plasmid DNA, e.g. with a relatively large size of at least 5.5 kbp. In embodiments, the nucleic acid is transfected into cells in the presence of saponin GE1741 and/or saponin SO1861. In embodiments, the nucleic acid is combined with poly-lysine, forming nanoplexes for transfection of the nucleic acid into a In cell. A third aspect of the invention relates to a kit of parts for delivering a nucleic acid encoding for a CRISPR/Cas construct into a cell in vitro. In addition, the invention also relates to a nanoparticle suitable for in vitro delivery of a nucleic acid into a cell, the nanoparticle comprising or consisting of a nucleic acid encoding for a CRISPR/Cas construct, a poly-lysine peptide and optionally a saponin.

### TECHNOLOGICAL FIELD

A first aspect of the invention relates to the use of a saponin in the in vitro delivery of a relatively large nucleic acid into a cell, wherein the nucleic acid comprises more than 5.5 kilo base-pairs (kbp). A second aspect of the invention relates to a method for delivering such a nucleic acid encoding for a CRISPR/Cas construct into a cell in vitro, in the presence of a saponin. A third aspect of the invention relates to a kit of parts for delivering a nucleic acid encoding for a CRISPR/Cas construct into a cell in vitro, in the presence of a saponin. In addition, the invention also relates to a nanoparticle suitable for in vitro delivery of a nucleic acid into a cell, the nanoparticle comprising or consisting of a nucleic acid encoding for a CRISPR/Cas construct, a poly-lysine peptide and optionally a saponin.

### BACKGROUND OF THE INVENTION

The CRISPR (clustered regularly interspaced short palindromic repeats)-Cas9-technology—often called “gene scissor”—is a tool, which revolutionized the field of gene technology in the last few years. With its simple, cheap and fast handling it is possible, to precisely cut out or insert DNA-sequences or even genes from the genome (genome editing). These properties make CRISPR-Cas9 to a valuable and promising technique for the gene therapy, outclassing other already existing gene editing tools. The genuine purpose of CRISPR-Cas9 is an antiviral defense mechanism of the bacteria Streptococcus pyogenes against bacteriophages.

CRISPR-Cas9 consists of three components. Cas9 is a bacterial enzyme, which induces strand breaks (nuclease, if double strand breaks; nickase, if single strand breaks). For the nuclease to recognize the location of its enzymatic activity, a trans-activating CRISPR RNA (tracrRNA) and the CRISPR-RNA (crRNA) form a guide-RNA (gRNA), which gives the Cas9-enzyme the necessary information for a precise cleavage.

During a bacteriophage infection, the crRNA possesses or obtains sequences of the phage sequences and with that the information to induce a double strand break and prevent an infection: While the tracrRNA's purpose is to stabilize the crRNA and together form a CRISPR-Cas9-complex, the Cas9-nuclease can cut the phage sequence after the annealing of the crRNA-sequence to its target. Cellular repair mechanisms subsequently lead to mutation, resulting in a gene inactivation (knock-out).

This discovered defense mechanism set a revolution of genomic editing in motion, when the idea arose to target selected genes by designing artificial crRNA. The CRISPR-Cas9-based knock-out or insertion (‘knock-in’) of genes was possible and distinguished itself from other genome editing techniques by its simple, fast and cost-effective applicability.

The introduction of the Cas9-Nuclease together with the gRNA into the cytosol is possible in form of the DNA or RNA, coding for the enzyme. However, transfection efficiency is poor and use of the Cas9 nuclease in vitro by introducing the (plasmid) DNA encoding for Cas9, is hampered due to low transfection efficiency of relatively large DNA constructs such as large plasmid DNA. Therefore, usually, the Cas9-nuclease/protein itself is being delivered with the help of commercially available reagents (for example Lipofectamine™ CRISPRMAX™ Cas9 Transfection Reagent), as with this way a more efficient transfer as well as less off-target effects are shown, and by in vitro delivering protein into the cells, poor DNA transfection efficiency is prevented.

To the knowledge of the inventors, so far no in vitro non-viral transfection enhancer exists, which is able to deliver relatively large, i.e. of DNA-constructs like Cas9-DNA-plasmids, i.e. plasmid DNA encoding for Cas9 and having a size of at least 5.5 kilo base pairs (kbp), in a simple and efficient way. Further, a universal transfection enhancer, which can deliver DNA such as plasmid DNA is not available to those working in the field of in vitro Cas9-DNA deliveries into cells.

### SUMMARY

The inventors surprisingly show that efficient in vitro delivery of relatively large plasmids, like for instance CRISPR/Cas9-nucleic acid constructs, can be established by applying plant defence molecules, i.e. saponins during transfection of the cells. Specific group of triterpenoid saponins (bisdesmosidic triterpenoid 12,13-dehydrooleanane-type saponin) such as SO1861 and GE1741 are able to increase the transfection efficiency of oligo-lysine-based nanoplexes in a considerable degree (Sama et aL. 2017; Sama et aL. 2018). Now, with the current invention the inventors established that: (i) the feasibility of the saponin-based transfection of the peptide-based nanoparticles comprising a nucleic acid and a poly-lysine (coined as sapofection by the inventors), (ii) the universal complexation of nucleic acids such as DNA, mRNA and minicircle-DNA, as well as (iii) the use of non-toxic concentrations for the saponins, i.e. 1 μg/mL-5 μg/mL, make the saponins a suitable constituent for DNA-comprising compositions applied in the field of large plasmid DNA transfections.

A first aspect of the invention relates to use of a saponin being a bisdesmosidic triterpenoid 12,13-dehydrooleanane-type saponin, in particular according to formula (I) in the in vitro delivery of a nucleic acid into a cell:

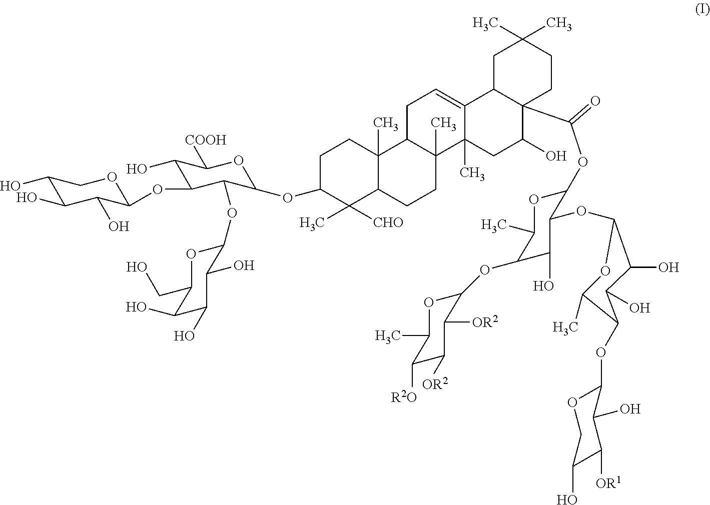

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin,
wherein the nucleic acid at least comprises more than 5.5 kilo base-pairs (kbp), and for example, comprises a first part of the nucleic acid that encodes for a CRISPR-associated endonuclease (Cas) and further optionally comprises a second part of the nucleic acid that encodes for a guide RNA (gRNA) or is a gRNA or comprises a gRNA.

The inventors have previously shown that plant secondary metabolites from the carnation family increase the intracellular delivery of relatively small-sized plasmid-DNA (<5 kbp), mini-circle-DNA (˜3 kbp), as well as mRNA (<1 kbp). The DNA transfection techniques using triterpene saponins was coined as sapofection, indicating that saponins are being used as tool to improve the transfection process. A drawback of commonly employed transfection reagents other than saponins is their low efficiency when relatively large plasmids such as CRISPR/Cas9-constructs have to be transfected into a target cell. The current inventors now show that plant secondary metabolites from Gypsophila elegans M. Bieb and Saponaria officinalis L. improve the intracellular delivery of large nucleic acid constructs, such as CRISPR/Cas9-constructs (for example plasmid DNA with a size >8.000 bp). The inventors surprisingly established that these saponin compounds being bisdesmosidic triterpenoid 12,13-dehydrooleanane-type saponin, e.g. GE1741 and SO1861, contribute to an effective delivery of complexed CRISPR/Cas9-constructs into eukaryotic cells.

Triterpene saponins are plant-derived secondary metabolites that are synthesized by a considerable number of plants. The inventors have previously shown that characterized triterpene saponins such as SO1861 from Saponaria officinalis L., GE1741 from Gypsophila elegans M.Bieb and AG1856 from Agrostemma githago L. increase the transfection efficiency of (small-sized) plasmid-DNA, mini-circle-DNA, as well as mRNA, containing oligo-lysine-based nanoplexes in a considerable degree (Clochard et al., 2020; Sama et al., 2018a; Sama et al., 2017). The simple feasibility of these transfections make triterpene saponins a novel and promising player in the field of transfections.

The CRISPR (clustered regularly interspaced short palindromic repeats)-Cas9-technology allows the precise deletion of genes within the chromosomal DNA as well as the insertion of DNA sequences into the genome. CRISPR/Cas9 consists of three components. Cas9 is an endonuclease, which induces strand breaks. The tracerRNA (trRNA) and the CRISPR-RNA (crRNA) direct the Cas9-nuclease to the desired cleavage site. The number of non-viral transfection reagents, which are suitable for the delivery of relatively large Cas9-DNA-plasmid constructs (plasmid DNA size is 7.000 bp or larger) is limited or even non-existent.

The current inventors now showed that GE1741 and SO1861 are potent facilitators and tools for the delivery of large nucleic acids including CRISPR/Cas9-DNA-plasmid constructs into eukaryotic cells. In addition, the inventors also showed that efficient knock-out experiments were successfully established with a GFP-donor sequence transfected as part of a large plasmid DNA (size larger than 7.000 bp), when transfection was in the presence of a saponin such as GE1741.

A second aspect of the invention relates to a method for delivering a nucleic acid comprising more than 5.5 kilo base-pairs (kbp), for example encoding for a CRISPR/Cas construct, into a cell in vitro, comprising the steps of:
(i) providing the nucleic acid and providing a saponin according to formula (I):

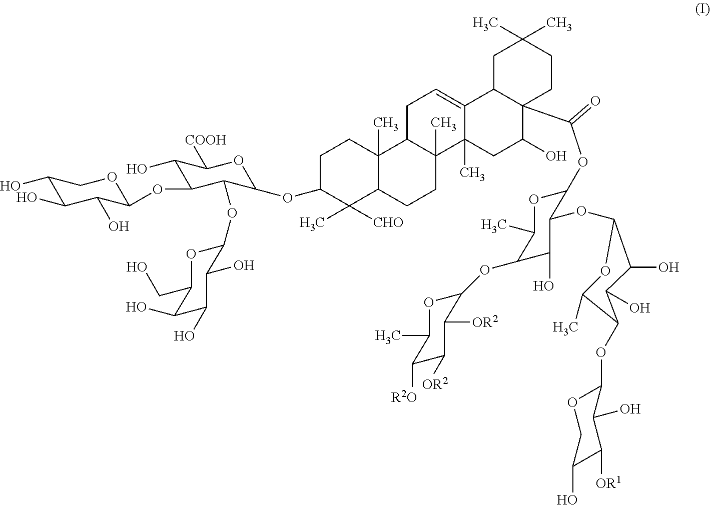

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin; and
(ii) incubating the cell with the nucleic acid in the presence of the saponin,

wherein, for example, the nucleic acid encoding for the CRISPR/Cas construct at least comprises a first part of the nucleic acid that encodes for a Cas and wherein the nucleic acid optionally comprises a second part of the nucleic acid that encodes for a gRNA or comprises a gRNA or is a gRNA.

For the complexation of the large plasmid DNA CMV-CAS9-2A-GFP (8.2 kbp) (Sigma-Aldrich, Taufkirchen, Germany), poly-lysine (K₁₆)-based nanoplexes were formulated (FIG. 1). The average size and PDI (poly dispersity index) were measured of P (peptide) D (Cas9GFP-plasmid) nanoplexes, formulated in different mass ratios using a Malvern Nano Zetasizer (Malvern Instruments Ltd, UK). With higher mass ratios a trend of lower sizes could be observed. The PDI showed constant monodisperse distribution, n≥3. The inventors also determined the complexation efficiency of the nanoplexes by applying agarose base gel electrophoresis and Quant-IT-PicoGreen assay as known in the art (Sama et al., 2017; FIG. 3). By applying the gel retention assay and quantitative complexation efficiency, the DNA retaining potential of PD-nanoplex formulations served as a qualitative measure of DNA complexation efficiency. The nanoplex formulations retained the DNA from a migration through the gel. The quantitative complexation assay showed an efficient complexation for all formulations comprising the plasmid DNA and the poly-lysine peptide.

After biophysical characterisation had established that nanoplexes of the large plasmid DNA and the poly-lysine peptide were formed, cell experiments were performed: transfections. Tests conducted by the inventors revealed toxic effects of nanoplexes towards cells at a peptide to plasmid DNA mass ratio of 16:1 mass ratio or higher, and in addition, further tests conducted by the inventors revealed an inefficient nucleic acid delivery with a peptide to plasmid DNA mass ratio of 2:1 or lower due to the peptide/DNA (N/P) ratio. Without wishing to be bound by any theory, the latter effect is most likely due to insufficient complexing of DNA. Therefore, the inventors demonstrate the benefits of the invention by examples conducted with PD-nanoplexes, formulated in a 4:1 ratio and in a 8:1 ratio. JIMT-1-cells (DSMZ no.: ACC 589) and Neuro-2A- cells (DSMZ no.: ACC 148) were transfected with nanoplexes containing relatively large Cas9-GFP-plasmid, with and without GE1741-Co-application or SO1861-Co-application at a non-toxic concentration, when the cell which is to be transfected is considered. The transfection efficiency of all transfection conditions was measured after 48 h incubation time using flow cytometry by comparison to the negative control in terms of fluorescence intensity (for example, cells contacted with the nanoparticles in the absence of saponin). The efficiency of GE1741-based transfections and SO1861-based transfections was also compared with efficiency of Lipofectamine3000™-based transfection of the nanoparticles. Transfections were performed according to protocols commonly known and applied in the field (Clochard et al., 2020; Sama et al., 2018a; Sama et al., 2017; Sama et al., 2018b). As the transfection efficiency did not increase from a 4.1 peptide/DNA ratio to the 8:1 ratio in Neuro-2A cells, a ratio of 4:1 was used for further tests and examples with JIMT-1 cells. Transfection efficiency of GE1741-mediated PD-transfection and SO1861-mediated PD-transfections on Neuro-2A cells and JIMT-1 cells was assessed. Cells were transfected with Cas9-GFP-DNA containing PD-nanoplexes, with and without GE1741 or SO1861 (final concentration of the saponin was 2 μg/ml). In Neuro-2A cells and JIMT-1 cells transection efficiency varied between 30% and 40%. Lipofectamin3000 served as reference. n≥3; a significant difference compared to the GE1741-free transfections or SO1861-free transfections was apparent, U-test, p≤0.05.

Compared to SO1861, GE1741 exhibited slightly better transfection modulating properties. For further knock-out experiments, GE1741 and Neuro-2A cells were tested. The integration of a linear donor sequence (LDS) containing the gene for GFP into the genomic DNA was assessed using three kits from Origene (Herford, Germany): The Slcl9a3-KN2.0 (CAT #: KN515911), the Slc25a24-KN2.0 (CAT #: KN515970) and the Slc26a4-KN2.0 (CAT #: KN516006), which kits all three were mouse gene knock-out kits via CRISPR, non-homology mediated. The kits contained all-in-one plasmids, coding for Cas9 and the target specific sgRNAs. The Slc26a4 kit targeted the solute carrier family 26, member 4 in the chromosome 12. The indel efficiency by targeting the locus Slc26a4 could be already shown in Neuro-2A cells (Ryu et al., 2018), and for the linear-donor-kits (Slc19a3, Slc25a24) two different sgRNA-sequences were tested. In the Examples section, FIG. 9 demonstrates the transfection efficiency in the presence of GE1741. A GFP-mediated fluorescence shows the efficient delivery and the integration of the linear donor sequence into the chromosomal DNA, under influence of the presence of the saponin. Thus, for the knock-out of different solute carrier genes (Sic) in Neuro-2A-cells via CRISPR/Cas9, neuro-2A-cells were transfected with different kits in order to compare the efficiency of different targets (knocked-out gene) and different integration techniques (see FIG. 8 and FIG. 10). A significant increase of transfection efficiency was observed for GE1741-treated cells, compared to the GE1741-free transfections, U-test, p≤0.05 (n≥3).

Thus, these examples surprisingly established that a bisdesmosidic triterpenoid 12,13-dehydrooleanane-type saponin, in particular saponin GE1741 from Gypsophila elegans M.Bieb and SO1861 from Saponaria officinalis L., is suitable for the efficient delivery of relatively large DNA with a size of 6.5 kbp or larger, up to 8.2 kbp or more, for example CRISPR/Cas9 constructs (e.g. plasmid DNA) into eukaryotic cells in a simple and efficient way.

A third aspect of the invention relates to a kit of parts for delivering a nucleic acid comprising more than 5.5 kbp, preferably more than 8 kbp, such as for example encoding for a CRISPR/Cas construct, into a cell in vitro comprising:
a first combination comprising or consisting of:
(a) a first container comprising a first plasmid DNA, for example, encoding for Cas9;
(b) optionally a second container comprising a second plasmid DNA, for example, encoding for gRNA;
(c) a third container comprising poly-lysine K₁₆ peptide;
(d) a fourth container comprising at least one bisdesmosidic triterpenoid 12,13-dehydrooleanane-type saponin, preferably one of: GE1741 and SO1861;
(e) instructions for use,
wherein the use at least comprises the preparation of nanoplexes of the poly-lysine K₁₆ peptide and the first plasmid DNA, and optionally the saponin, wherein the first plasmid has a size of at least 5.5 kbp, or
a second combination comprising or consisting of:
(a) a first container comprising a third plasmid DNA, for example encoding for Cas9 and encoding for gRNA;
(b) a second container comprising poly-lysine K₁₆ peptide;
(c) a third container comprising at least one bisdesmosidic triterpenoid 12,13-dehydrooleanane-type saponin, preferably one of: GE1741 and SO1861;
(d) instructions for use,
wherein the use at least comprises the preparation of nanoplexes of the poly-lysine K₁₆ peptide and the third plasmid DNA, and optionally the saponin,
wherein the third plasmid has a size of at least 5.5 kbp,
or
a third combination comprising or consisting of:
(a) a first container comprising the first plasmid DNA, for example encoding for Cas9;
(b) optionally a second container comprising an oligonucleotide, like an RNA oligonucleotide for example a gRNA RNA oligonucleotide;
(c) a third container comprising poly-lysine K₁₆ peptide;
(d) a fourth container comprising at least one bisdesmosidic triterpenoid 12,13-dehydrooleanane-type saponin, preferably one of: GE1741 and SO1861;
(e) instructions for use,
wherein the use at least comprises the preparation of nanoplexes of the poly-lysine K₁₆ peptide and the first plasmid DNA, and optionally the saponin,
wherein the first plasmid has a size of at least 5.5 kbp.

The invention also relates to a composition comprising a nanoparticle, suitable for in vitro delivery of a nucleic acid, for example encoding for a CRISPR/Cas construct, into a cell, wherein the nanoparticle comprises or consists of:
(i) the nucleic acid comprising more than 5.5 kilo base-pairs (kbp), for example encoding for a CRISPR/Cas construct;
(ii) a poly-lysine peptide, and
(iii) a saponin being a bisdesmosidic triterpenoid 12,13-dehydrooleanane-type saponin, possibly, wherein the nucleic acid encoding for a CRISPR/Cas construct at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA.

Preferred compositions are the composition comprising a nanoparticle, suitable for in vitro delivery of a nucleic acid encoding for a CRISPR/Cas construct into a cell of the invention, further comprising a saponin according to formula (I):

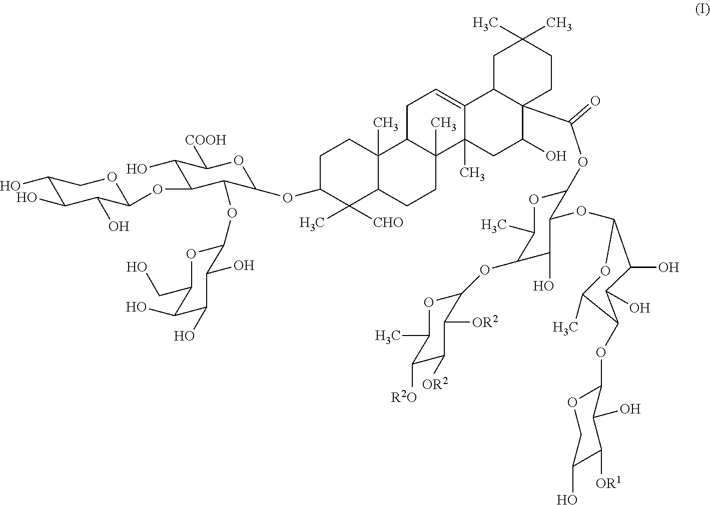

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin.

The nucleic acid encoding for a CRISPR/Cas construct at least comprises a first part of the nucleic acid that encodes for a CRISPR-associated endonuclease (Cas) and the nucleic acid optionally comprises a second part of the nucleic acid that encodes for a guide RNA (gRNA) or is a gRNA or comprises a gRNA. Preferably, the saponin is GE1741 or SO1861, more preferably GE1741. Preferably, the poly-lysine peptide is consisting of 16 lysine residues. Preferably, the nucleic acid is provided as part of a plasmid DNA. Preferably, the encoded Cas is Cas9 (SEQ ID NO: 1) or a Cas which has at least 90% sequence identity with the Cas9.

The present invention will be described with respect to particular embodiments but the invention is not limited thereto but only by the claims. The embodiments of the invention described herein can operate in combination and cooperation, unless specified otherwise.

### Definitions

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a composition, method, use, kit of parts, listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

### BRIEF DESCRIPTION OF THE DRAWINGS

FIG. 1. Table displaying the average size and PDI of PD-nanoplexes, formulated in different mass ratios. Peptide/DNA (PD)-nanoplexes were formulated in different mass ratios, in order to assess an optimal formulation with low particle size and preferably monodisperse size distribution. With higher mass ratios a trend of lower sizes was observed. The PDI showed constant monodisperse distribution. n≥3

FIG. 2A-C. Size distribution of DNA-loaded (D) oligo-lysine-based (P) nanoplexes. PD-nanoplexes (4:1 mass ratio for peptide mass:DNA mass), loaded with Cas9-DNA (A), GFP-DNA (B) or GFP-Cas9-DNA (C), showed a dominant size distribution around 120 nm. The share of aggregated particles was low (small peak in the 1.000-10.000 nm size range).

FIG. 3A, 3B. Gel retention assay for a series of nanoplex formulations incl. quantitative complexation efficiency. The DNA retaining potential of PD-nanoplex formulations served as a qualitative measure of DNA complexation efficiency. The formulated nanoplexes were applied into the agarose gel pockets, subsequently a voltage was applied. Well complexed nanoplexes show low ethidium bromide staining and no migration on the gel. Compared to the free migrating DNA (left lane in FIG. 3A and FIG. 3B; 0:1) none of the nanoplex formulations did leave their pockets in any of the second—eighth lanes from left to right. The signal of PD (GFP-DNA; last three lanes, lanes sixth-eighth from the left in FIG. 3A and FIG. 3B) appeared to be weaker, indicating a better complexation of the smaller DNA. The quantitative complexation assay showed an efficient complexation for all formulations. GFP-loaded nanoplexes were the best complexed, larger-sized plasmids (Cas9-GFP-DNA; FIG. 3B, lanes second-fifth from the left) were slightly less complexed, and complexation efficiency for PD(Cas9-DNA) plasmid (FIG. 3A, lanes second-fifth from the left) was about in between those other two complexation efficiencies.

FIG. 4A, B. Transfection efficiency of saponin-mediated PD (Cas9-GFP)-transfections on Neuro-2A cells (FIG. 4A) and on JIMT-1-cells (FIG. 4B). Neuro-2A cells (A) and JIMT-1 cells (B) were transfected with GFP-DNA (i.e. a plasmid comprising the GFP DNA) bearing PD-nanoplexes or with Cas9-GFP-DNA bearing PD-nanoplexes, with and without the highest non-toxic saponin Co-administration (GE1741 Co-administration at 2 μg/ml or SO1861 Co-administration at 2 μg/ml). While GFP-DNA-transfections reached high efficiencies of around 70%, approximately 40% of all cells expressed GFP after Cas9-GFP-transfection. Lipofectamin3000 served as positive control and showed only an effective GFP-DNA-delivery. GFP-Cas9-DNA was delivered only in low amount. N≥3; *: significant difference compared to the saponin-free transfection (only nuclease), U-test.

FIG. 5A-F. Confluence measurements of Neuro-2A-cells during PD (Cas9-GFP)-transfections ±saponin-administration. A visual monitoring of confluence was conducted during the Cas9-GFP-transfections (FIGS. 5A, E, F) and GFP-DNA-transfections (FIGS. 5A, C, D) of Neuro-2A-cells. The display of confluence during the incubation time was provided by algorithms of the Cytosmart. No distinct toxic effects could be observed compared to the untreated negative control (FIGS. 5A, B). Saponin GE1741 was absent (FIGS. 5A, C, E) or present (FIGS. 5A, D, F) during the confluency assessment over time. FIG. 5B-F show the Neuro-2A cell-confluency after an incubation time of 95 h.

FIG. 6A-E. GFP-Knockout of GFP-expressing Neuro-2A-GFP-cells via PDD (Cas9-DNA, gRNA (GFP)-DNA)-nanoplexes±saponin administration (co-transfection). GFP expressing Neuro-2A-cells were transfected via co-transfection of Cas9-DNA-plasmid and gRNA (GFP)-DNA-plasmid in a ratio of 4:0.5:0:5, with 500 ng of each DNA/well and 4.000 ng poly-lysine K₁₆ peptide perwell. A decrease of the FITC-H median was observed for transfections with and without saponins GE1741. With saponin-Co-administration, the FITC-H was decreased by 10% (FIG. 6D) and the amount of cells not considered “fluorescent” compared to negative control, was increased by 7.5% (FIG. 6E).; n 3. FIG. 6A: FACS results with negative control; FIG. 6B: FACS results with PDD(Cas9-DNA, gRNA(GFP)-DNA), 4:1; FIG. 6C: FACS results of PDD(Cas9-DNA, gRNA(GFP)-DNA), 4:1+GE1741; FIG. 6D: values for the median FITC-H for the test samples of FIG. 6A-C and the control Lipofectamine3000DD(Cas9-DNA); FIG. 6E: amount of non-fluorescent cells in the test samples of FIGS. 6A-C and the control Lipofectamine3000DD(Cas9-DNA).

FIG. 7A-E. GFP-Knockout of GFP-expressing Neuro-2A-cells via PD (Cas9-gRNA (GFP)-DNA)-nanoplexes±saponin administration (All-in-one-transfection). GFP expressing Neuro-2A-cells were transfected via “All-in-one”-transfection of a Cas9-gRNA (GFP)-DNA-plasmid in a ratio of 4:1, 500 ng of each DNA/well. No FITC-H could be observed (FIG. 7D), however, the amount of cells considered fluorescent was elevated by 11% in a PD mass ratio of 8:1 (FIG. 7E).; n≥3. FIG. 7A: FACS results with negative control; FIG. 7B: FACS results with PD(Cas9-gRNA(GFP)-DNA, 4:1; FIG. 7C: FACS results of PD(Cas9-gRNA(GFP)-DNA, 4:1+GE1741; FIG. 7D: values for the median FITC-H for the test samples of FIG. 7A-C and for PD(Cas9-gRNA(GFP)-DNA, 8:1 and PD(Cas9-gRNA(GFP)-DNA, 8:1+GE1741; FIG. 7E: amount of non-fluorescent cells in the test samples of FIG. 7A-C and for PD(Cas9-gRNA(GFP)-DNA, 8:1 and PD(Cas9-gRNA(GFP)-DNA, 8:1+GE1741.

FIG. 8. CRISPR-Cas9-based gene knock-out and reporter gene knock-in (OriGene). By conducting a Co-transfection of an All-in-One-DNA-plasmid plasmid DNA (top left) with Cas9-sequence and gRNA-sequence, together with a linear donor sequence (LDS) coding for GFP and coding for Puromycin (‘Linear donor’, comprising an EF1a (i.e. the EF1 alpha promotor), the GFP sequence, a P2A sequence (i.e. a 2A sel-cleaving peptide) and the Puromycin (‘Puro’) sequence), a gene knock-out and knock-in experiment is performed. A gene of a solute carrier (Slc) was selected as gRNA-target (‘Target sequence’). After Cas9-induced strand break (Slc knock-out), the LDS is integrated via non-homologous recombination—forward or reverse (GFP—Puromycin knock-in). A selection of the modified cell clone is achieved via the inserted puromycin resistance gene.

FIG. 9A-D Transfection efficiency of saponin-mediated PDD (Cas9-gRNA (Slc26a4)-DNA, LDS-DNA)-transfections. A: Neuro-2A-cells were transfected with PDD-nanoplexes (Cas9-gRNA (Slc26a4)-DNA, GFP-Puromycin-LDS, 4:1 and 8:1 mass ratio when the mass of the peptide and the DNA are considered), with and without GE1741-Co-administration. The application of GE1741 led to a transfection efficiency of 20% and 30%, respectively, for the 4:1 and 8:1 mass ratio. Lipofectamin3000 served as positive control. B-D: Distinct fluorescence intensity increase of Neuro-2A-cells after saponin-mediated GFP-gene knock-in. n≥3; *: significant difference compared to the saponin-free transfection (only nuclease), U-test was performed. FIG. 9B: negative control; FIG. 9C: PDD(Cas9-gRNA(Slc26a4)-DNA, Puro-GFP-LDS), 4:1); FIG. 9D: PDD(Cas9-gRNA(Slc26a4)-DNA, Puro-GFP-LDS), 4:1)+GE1741.

FIG. 10(1-3). Reporter gene Knock-In techniques—homologous recombination vs. non-homologous recombination. FIG. 10 displays the knock-in technique applying homologous recombination using a circular donor template DNA, as part of a DNA vector, here pUC. For comparison, FIG. 8 displays the knock-in technique applying non-homologous recombination using an LDS, a linear donor template DNA for knocking-in a nucleic acid of interest. The homologous recombination kit (1+2) consists of a circular DNA donor sequence (2: circular Donor template DNA containing homologous arms and functional cassette), and the plasmid DNA encoding for the Cas9 and the gRNA, as displayed in FIG. 8 (1: target sequence cloned in pCas guide vector), where the donor sequence is integrated via homologous repair (3: co-transfection of 1, the pCas guide plasmid DNA+2, the pUC vector serving as the circular donor template DNA bearing plasmid DNA, results in genome incorporation of the donor sequence (GFP-Puro(mycin)), providing the edited chromosome with knocked-in gene. The target gene is knocked out, the GFP is under the native gene promotor, and the puromycin gene (Puro) is under the PGK promotor in this example. While the circular donor template DNA (2) is less degradable than a linear donor sequence (See e.g. FIG. 8, top right), the integration process appears to be a more complex process, as apparent when gene integration efficiency is assessed by measuring the extent of GFP fluorescence.

FIG. 11A, B. Knockout of different solute carrier (Sic) in Neuro-2A-cells—homologous recombination vs. non-homologous recombination. Neuro-2A-cells were transfected using NHR kits and using HR kits in order to compare the efficiency for different targets and different integration techniques, under influence of the absence or presence of saponin GE1741 in the K₁₆-peptide comprising nanoplexes. While all NHR-knock-in kits (Slc26a4, Slc19a3, and Slc25a24) showed a significant increase of transfection efficiency, no GFP expression could be observed with HR-knock-in kits. n 3; *: significant difference compared to the saponin-free transfection (only nuclease), U-test. (A) co-transfection of plasmid DNA comprising nucleic acids encoding for Cas9 and gRNA(Slc26a4), and LDS DNA encoding for puromycin and green fluorescent protein (Puro-GFP-LDS). (B) co-transfection of plasmid DNA comprising nucleic acids encoding for Cas9 and gRNA(Slc), and donor template DNA encoding for puromycin and green fluorescent protein (Puro-GFP-DS) provided as DNA vector.

FIG. 12A, B. Puromycin selection after Knock-In of the GFP-Puromycin-LDS. After the knock-out of solute carrier Slc26a4 and integration of GFP-Puromycin-Donor sequence, the cells were cultivated for seven passages until Puromycin selection was started. Different concentrations of Puromycin were applied and the cell viability was observed (A, B). 1 μg/mL and 2 μg/mL showed a decrease of confluence with a number of surviving cell populations, due to the Puromycin resistance. (B) Green fluorescence is apparent as whitening in the wells of the displayed cell culturing plate. Plate wells in rows A-C are numbered 1-4 from left to right. Wells C3-4 were empty.

FIG. 13, FITC-H analysis after isolation of Cas9-mediated Slc26a4-knock-out-GFP-knock-in cells. The grown Neuro-2A-cell clones were trypsinized and applied to flow cytometry. The cells were analyzed in terms of fluorescence (FITC-H). Untreated Neuro-2A-cells and Neuro-2A-GFP-cells were used as negative and positive control, respectively. Two types of cell clones were observed. While some clones showed no fluorescence and indicated no stable GFP expression, other clones showed two cell populations.

### DETAILED DESCRIPTION OF THE INVENTION

The present invention will be described with respect to particular embodiments but the invention is not limited thereto but only by the claims. The embodiments of the invention described herein can operate in combination and cooperation, unless specified otherwise.

It is a first goal of the present invention to provide an improved method for in vitro delivery of a relatively large nucleic acid such as a plasmid DNA, for example with a size of 5.5 kbp or larger, into a cell.

It is an objective of the current invention to provide a method for in vitro providing a target cell with a relatively large nucleic acid, preferably at least encoding for a Cas (i.e. a nucleic acid with a size of at least 5.5 kbp), which method is accompanied by less cumbersome manufacturing requirements and improved nucleic acid transfection efficacy, for example when method steps for preparing the composition comprising the nucleic acid is concerned and/or when the relative transfection efficiency is concerned when transfection according to the invention is compared to transfection methods commonly applied in the field. It is also an objective of the current invention to provide tools such as a kit, applicable for combining components of the kit with a selected nucleic acid for the purpose of in vitro delivery (transfection) of the nucleic acid into a target cell. Such a nucleic acid is typically larger than 5.000 bp, and even as large as 8.000-9.000 bp or larger.

At least one of the above objectives is achieved by providing an in vitro method for providing a target cell with a relatively large nucleic acid, preferably at least encoding for a Cas of the invention (i.e. a nucleic acid with a size of at least 5.5 kbp). At least a further objective is achieved by providing a kit of the invention comprising transfection efficacy enhancing reagents such as saponin GE1741 and/or saponin SO1861.

The present invention will be described with respect to particular embodiments but the invention is not limited thereto but only by the claims. While the invention has been described in terms of several embodiments, it is contemplated that alternatives, modifications, permutations and equivalents thereof will become apparent to one having ordinary skill in the art upon reading the specification. The invention is not limited in any way to the illustrated embodiments. Changes can be made without departing from the scope which is defined by the appended claims.

A first aspect of the invention relates to use of a bisdesmosidic triterpenoid 12,13-dehydrooleanane-type saponin in the in vitro delivery of a relatively large, i.e. with a size of at least 5.5 kbp or larger like at least 8 kbp, nucleic acid into a cell: As used herein, the term “saponin” has its regular established in the art meaning and refers herein to a group of amphipathic glycosides which comprise one or more hydrophilic saccharide chains covalently attached to a lipophilic skeleton structure termed the aglycone core or sapogenin. The saponin may be naturally occurring or synthetic (i.e. non-naturally occurring). As used herein, the term “saponin” is to be construed as including naturally-occurring saponins as well as saponins synthesized de novo through chemical and/or biotechnological synthesis routes. A bisdesmosidic triterpenoid 12,13-dehydrooleanane-type saponin has a triterpene, i.e. pentacyclic C30 terpene aglycone core that is of 12,13-dehydrooleanane type and has two sugar chains attached thereto (i.e. is bidesmosidic).

A particularly advantageous aspect of the invention relates to use of a saponin in the in vitro delivery of the relatively large, i.e. with a size of at least 5.5 kbp or larger like at least 8 kbp, nucleic acid into a cell, wherein the saponin is according to formula (I):

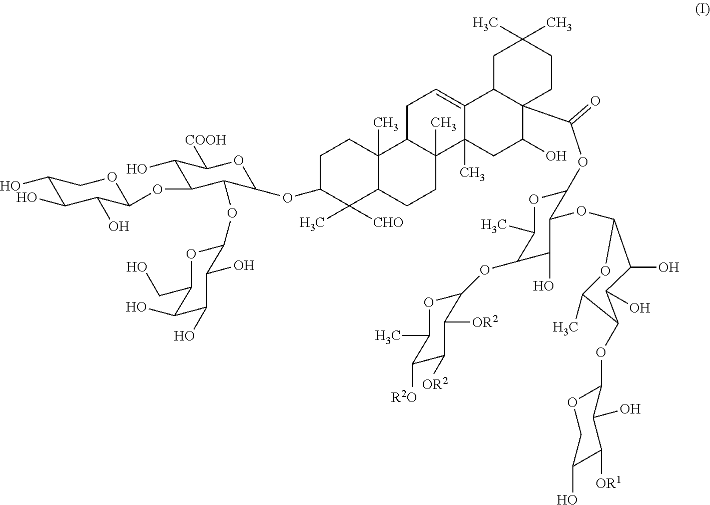

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin.

Typically, the use of the invention is characterized in that the nucleic acid comprises more than at least 6 kbp, preferably at least 7 kbp, more preferably at least 7.5 kbp, most preferably at least 8 kbp, or even at least 9 kbp or 10 kbp or more, and preferably is a plasmid DNA, for example, CRISPR-associated endonuclease (Cas)-encoding plasmid DNA.

Advantageously, the use of the invention is characterized in that the nucleic acid comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA, preferably wherein the nucleic acid is a Cas-encoding plasmid DNA, more preferably in that the nucleic acid is a plasmid DNA for expressing at least the Cas, even more preferably in that the nucleic acid is a plasmid DNA for expressing the Cas and for expressing the gRNA.

Hence, a specific aspect of the invention relates to use of a saponin according to formula (I) in the in vitro delivery of a nucleic acid into a cell:

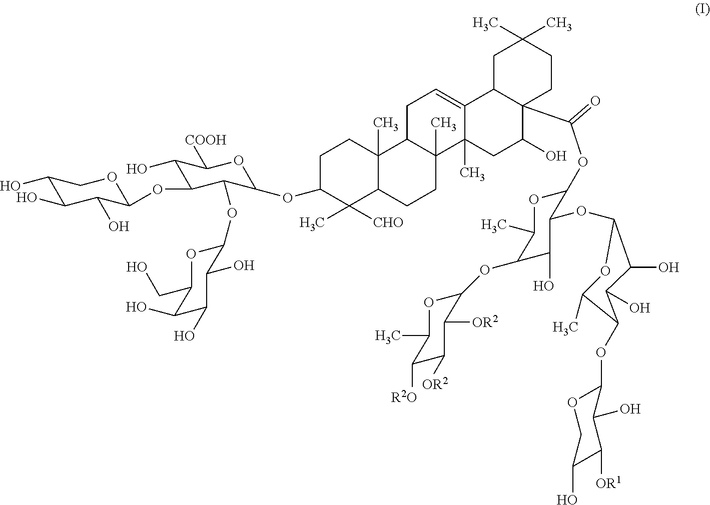

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin,

wherein the nucleic acid at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA.

Typically, the use of the invention is characterized in that the cell is a eukaryotic cell.

The use according to the invention is preferably characterized in that R¹ of the saponin according to formula (I) is a xylose residue and/or in that the saponin carries exactly two acetyl groups.

Preferred is the use according to the invention, characterized in that the acetyl groups of the saponin according to formula (I) are bonded to the oxygen atoms in C3 position and in C4 position of the corresponding quinovose residue of the saponin.

Also preferred is the use according to the invention, characterized in that one of the R² residues of the saponin according to formula (I) is a xylose residue, one of the R² residues is an acetyl group and one of the R² residues is H.

According to some embodiments, the use according to the invention is characterized in that the xylose residue of the saponin according to formula (I) is bonded to the oxygen atom in C3 position of the corresponding quinovose residue and in that the acetyl group is bonded to the oxygen atom in C4 position of the corresponding quinovose residue of the saponin.

Preferred is the use according to the invention, characterized in that R¹ of the saponin according to formula (I) is a xylose residue and in that two R² groups of the saponin according to formula (I) are acetyl groups which are bonded to the oxygen atoms in C3 position and in C4 position of the corresponding quinovose residue, and in that the third R² group is H.

Particularly preferred is the use according to the invention, characterized in that the saponin is GE1741 according to formula (II):

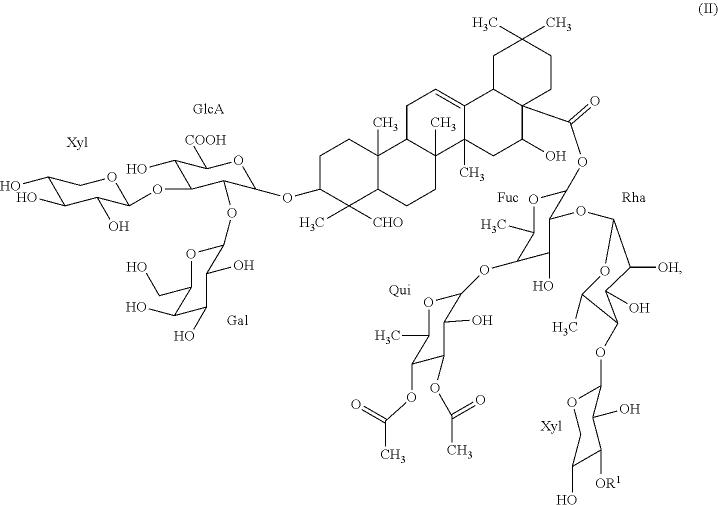

wherein R¹ is xylose;
or in that the saponin is SO1861 according to formula (111):

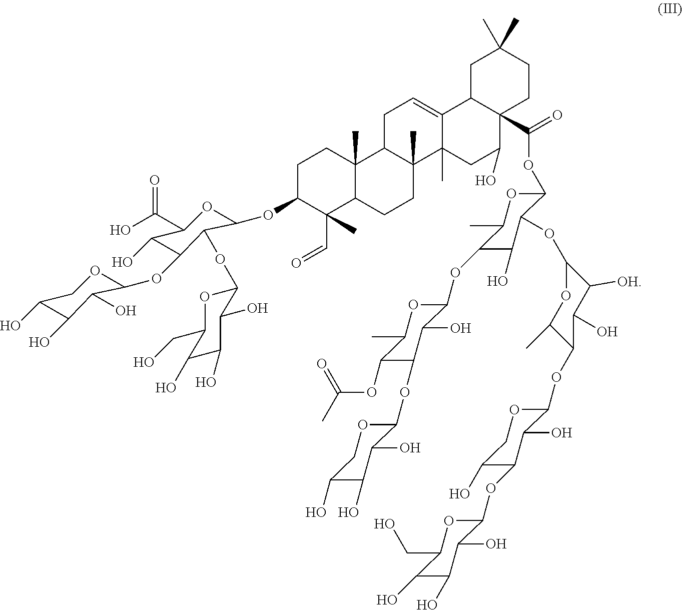

The use of the invention may be advantageously be characterized in that the nucleic acid forms part of a nanoparticle, which nanoparticle further comprises a nanoparticle-forming compound, preferably wherein the nanoparticle-forming compound comprises or consists of a poly-lysine peptide, preferably wherein the poly-lysine peptide consists of 5-25 lysine residues, more preferably the poly-lysine peptide consists of 16 lysine residues. In a particular embodiment, the use may be characterized in that the nanoparticle comprises the nanoparticle-forming compound and the nucleic acid in a mass ratio in a range of 3:1 to 15:1, preferably in a range of 3:1 to 9:1.

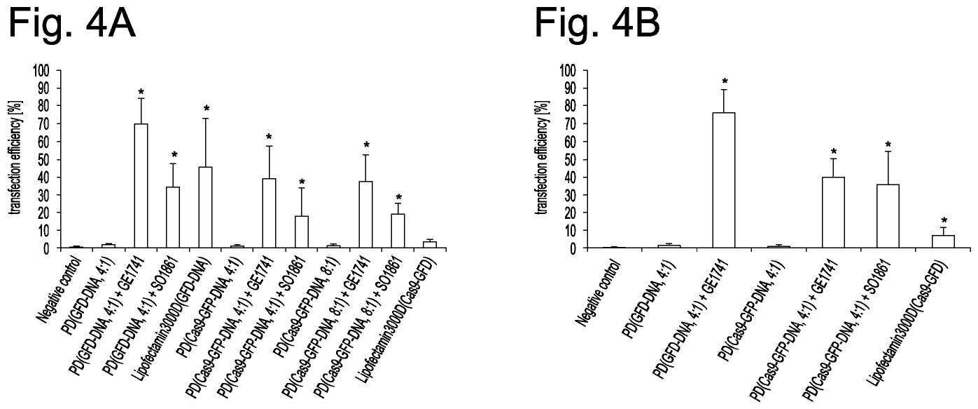

By combining saponin SO1861 or GE1741, preferably GE1741, with plasmid DNA that is nanoplexed with poly-lysine peptide (the saponin based ‘sapofection’ technology, relating to nanoplexed structures, i.e. nanoplexes, consisting of poly-lysine peptide, preferably K₁₆ peptide, nucleic acid, preferably in the form of plasmid DNA, and saponin, preferably SO1861 or GE1741, more preferably GE1741, the inventors were surprisingly able to effectively deliver into a cell in vitro a relatively large nucleic acid in the form of a DNA-plasmid (plasmid DNA with a size of >6.500 bp, such as 7.0 kbp-8.3 kbp), here for example coding for Cas9 protein and incorporated into peptide-based (poly-lysine K₁₆ peptide) nanoplexes, for example in mammalian cells (e.g. JIMT-1 cancer cells) and murine cancer cells (Neuro-2A). The saponin such as SO1861 or GE1741 is mixed with pre-formed nanoplexes consisting of plasmid DNA and poly-lysine peptide, therewith providing a composition comprising the nanoplexes consisting of plasmid DNA and poly-lysine peptide, for example K₁₆ peptide, and comprising the saponin such as GE1741. Such a mixture is contacted with target cell which are to be transfected with the nucleic acid. Alternatively, a first composition containing the saponin and a second composition containing the nanoplexes are both contacted with the cells, without prior mixing of the first composition and second composition. Here, for example, the first composition and the second composition are added to the cell culture medium in which the cells selected for transfection by the nucleic acid are cultured. For example, the co-transfection of a double-strand DNA donor sequence, such as for example the donor sequence for green fluorescent protein (GFP), either or not combined with the donor DNA sequence for puromycin, which is inserted in the Cas9 cleavage site, showed efficient donor-sequence-derived gene expression. The transfection efficiency achieved with the transfection method of the invention and achieved with the use of the nanoplexes of relatively large DNA plasmid and poly-lysine, in the presence of saponin such as GE1741 and SO1861, as part of the composition comprising the nanoplex structures or added separately to the cells selected for transfection with the DNA plasmid, according to the invention, was higher than obtainable with current gold standard Lipofectamine3000™. Reference is for example made to the Examples section, FIGS. 4-7, 9, 11 and 12. As said, according to the invention, the saponin is either pre-mixed with the nanoparticles composed of poly-lysine and nucleic acid before the mixture is brought in contact with cells, or cells are transfected by simultaneous adding the nanoparticles and the saponin separately to the cells, for example by adding the nanoparticles and the saponin to the cell culture medium.

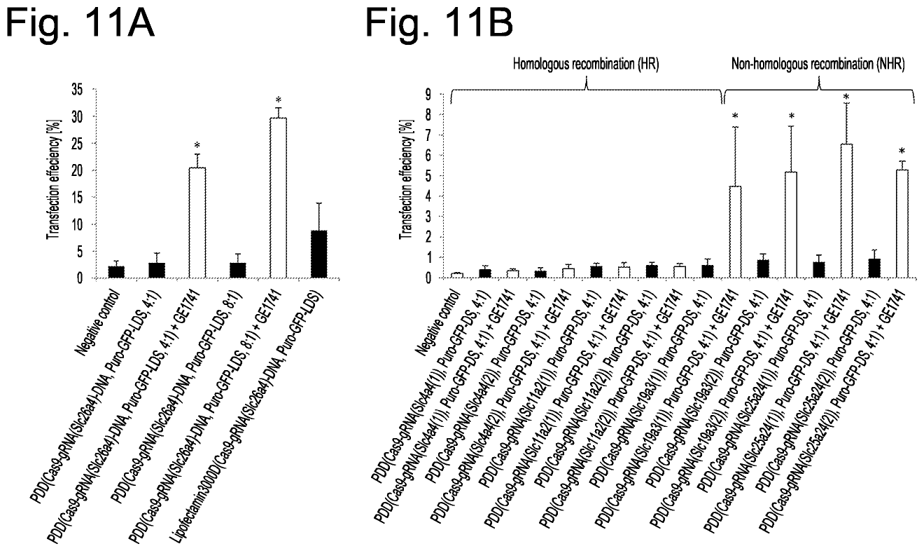

By applying the method of the invention and with the use according to the invention, the inventors achieved a transfection efficacy in vitro of at least 15%, when transfection of a plasmid DNA with a size of about 7 kbp or even 8.2 kbp is considered. Preferably, transfection efficiency is at least 20%, more preferably at least 25%, most preferably at least 30%. Indeed, by applying the method of the invention and with the use according to the invention, transfection efficiency of at least 30% was obtained (see also FIG. 11). Typically, such a high efficiency, which is for example at least three times higher than the transfection efficiency obtainable by transfecting plasmid DNA with the aid of e.g.

Lipofectamin3000, is achievable by transfecting plasmid DNA with a size of at least 7 kbp such as plasmid DNA encoding for at least a Cas enzyme such as Cas9 with an amino acid sequence according to SEQ ID NO:1, Cas12a with an amino acid sequence according to SEQ ID NO:2 or Cas13a with an amino acid sequence according to SEQ ID NO:3, preferably Cas9, under influence of the presence of the saponin during the transfection. Typically, the plasmid DNA also encodes for the gRNA. Typically, such a plasmid DNA has a size of at least 8 kbp, and still is efficiently transfected in vivo by applying the method of the invention and with the use according to the invention, due to the presence of the saponin during transfection. Alternatively, and evenly preferred is the use according to the invention wherein the nucleic acid encodes for the Cas such as Cas9, and wherein the gRNA is provided as a second nucleic acid encoding for the gRNA (second plasmid DNA encoding for the gRNA), or as a gRNA RNA sequence.

Typically, the use of the invention includes a nucleic acid encoding for Cas9 with an amino acid sequence according to SEQ ID NO:1, Cas12a with an amino acid sequence according to SEQ ID NO:2 or Cas13a with an amino acid sequence according to SEQ ID NO:3, and preferably encoding for said Cas9. In addition, also preferred is the use according to the invention, characterized in that the Cas encoding nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b).

An embodiment is the use of the invention, wherein the nucleic acid, preferably a plasmid DNA, at least comprises a first part of the nucleic acid that encodes for a CRISPR-associated endonuclease (Cas), preferably Cas9 (SEQ ID NO:1) or a Cas with at least 90% sequence identity with this Cas9, and wherein the nucleic acid optionally comprises any one or more of: a second part of the nucleic acid that is a non-coding guide RNA comprising CRISPR RNA (crRNA) and comprising trans-activating CRISPR RNA (tracrRNA), wherein the encoded guide RNA is a single-guide RNA or a two-piece RNA, wherein the tracrRNA comprises a binding site for the Cas encoded by the first part of the nucleic acid. Alternatively, an embodiment is the use of the invention wherein the Cas, such as the Cas9, is encoded by a first nucleic acid such as a first plasmid DNA, and the gRNA is encoded by a second nucleic acid such as a second plasmid DNA. Preferred is the use according to the invention wherein the nucleic acid encoding for the Cas and the nucleic acid encoding for the gRNA are both encompassed by a single nucleic acid such as a single plasmid DNA.

For the purpose of providing a cell with a (stably transfected) knocked-in gene encoding for a protein of interest, the use of the invention is preferred, wherein the in vitro delivery of a nucleic acid involves the delivery of the single plasmid DNA comprising the nucleic acids encoding for the Cas such as Cas9 and encoding for the gRNA, or the delivery of the two separate plasmid DNAs, the first one encoding for the Cas, the second one encoding for the gRNA, together with a further nucleic acid that is a DNA donor template, encoding for the gene selected for knocking in the genome of the cell. The donor template DNA is either a linear donor sequence, or a circular donor template DNA, such as a DNA vector. The inventors established that stable transfection and forming of knocked-in cells is more efficient when a linear DNA donor sequence is applied for the use of the method, compared to the application of a circular donor template DNA. Therefore, the use is preferred wherein the in vitro delivery of a nucleic acid into a cell relates to the in vitro delivery of a CRISPR/Cas construct into a cell, comprising the delivery of a linear donor sequence.

Preferred is the use according to the invention, characterized in that the Cas encoded by the in vitro delivered nucleic acid is Cas9 (SEQ ID NO:1), Cas12a (SEQ ID NO: 2), Cas13a (SEQ ID NO: 3), or wherein said nucleic acid is encoding for a Cas with at least 90% amino-acid residue identity with any one of these Cas9, Cas12a and Cas13a, or for an endonuclease with Cas-like endonuclease activity similar to endonuclease activity of any one of these Cas9, Cas12a and Cas13a, and capable of binding to the tracrRNA, when Cas9 is considered, and having at least 90% amino-acid residue identity with any one ofthese Cas9, Cas12a and Cas13a.

The inventors established that poly-lysine (10-20 lysine residues, typically 16 lysine residues) efficiently forms nanoparticles (nanoplexes) with nucleic acid such as plasmid DNA with a size of at least 5.5 kbp, such as at least 7 kbp or even at least 8 kbp. The inventors established that such nanoparticles are particularly efficiently transfected into cells in the presence of 1-5 microgram/mL saponin such as SO1861 or GE1741 during transfection of cells, when the mass ratio between the poly-lysine peptide and the nucleic acid is less than 16:1, such as 3:1 to 15:1. The inventors also established that for efficient nucleic acid transfection, the mass ratio in the nanoparticles, between the poly-lysine peptide and the nucleic acid, should be higher than 2:1, such as 3:1-15:1. It is preferred that with the use of the invention, the nucleic acid selected for in vitro delivery into a cell, is provided as part of a nanoplex (nanoparticle), wherein the nanoplex consist of the nucleic acid, preferably in the form of a plasmid DNA, typically with a size of at least 5.5 kbp, e.g. at least 6.0 kbp, such as 6-9 kbp and a poly-lysine peptide, such as K₁₂ peptide -K₂₀ peptide (a poly-lysine peptide consisting of any one of 12 to 20 lysine residues), preferably K₁₆ peptide. During transfection of cells with such nucleic acid, saponin is also added to the cells, or saponin is first pre-mixed with the nanoparticles and then added to the cells. Optional is the use of the invention, wherein the nucleic acid selected for in vitro delivery into a cell, is provided as part of a nanoplex (nanoparticle), wherein the nanoplex consist of the nucleic acid, preferably in the form of a plasmid DNA, typically with a size of at least 5.5 kbp, e.g. at least 6.0 kbp, such as 6-9 kbp, a saponin, such as SO1861 or GE1741, preferably GE1741, and a poly-lysine peptide, such as K₁₂ peptide -K₂₀ peptide (a poly-lysine peptide consisting of any one of 12 to 20 lysine residues), preferably K₁₆ peptide. Typically, in the use of the invention, the nanoparticle comprises the nanoparticle-forming compound such as the poly-lysine peptide and the nucleic acid, in a mass ratio in a range of 3:1 to 15:1, preferably in a range of 3:1 to 9:1, since at a mass ratio of 2:1 transfection is inefficient and since at a mass ratio of 16:1, the nanoparticles (nanoplexes) are toxic to the cells.

A second aspect of the invention relates to a method for delivering a nucleic acid comprising more than 5.5 kilo base-pairs (kbp) into a cell in vitro, comprising the steps of:
(i) providing the nucleic acid; and
(ii) providing a saponin that is a bisdesmosidic triterpenoid 12,13-dehydrooleanane-type saponin; and
(iii) incubating the cell with the nucleic acid in the presence of the saponin.

In a preferred embodiment of the second aspect of the invention, a method is provided wherein the saponin is according to formula (I):

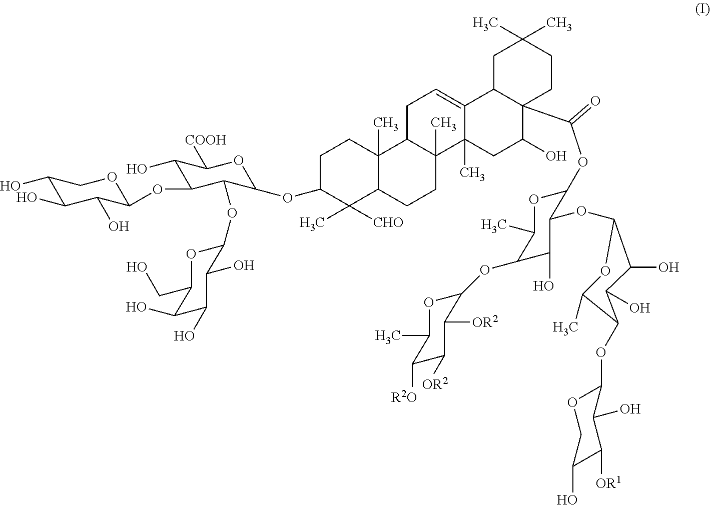

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin.

In a specific embodiment, the second aspect of the invention may relate to a method for delivering a nucleic acid encoding for a CRISPR/Cas construct into a cell in vitro, comprising the steps of:
(i) providing the nucleic acid and providing a saponin according to formula (I):

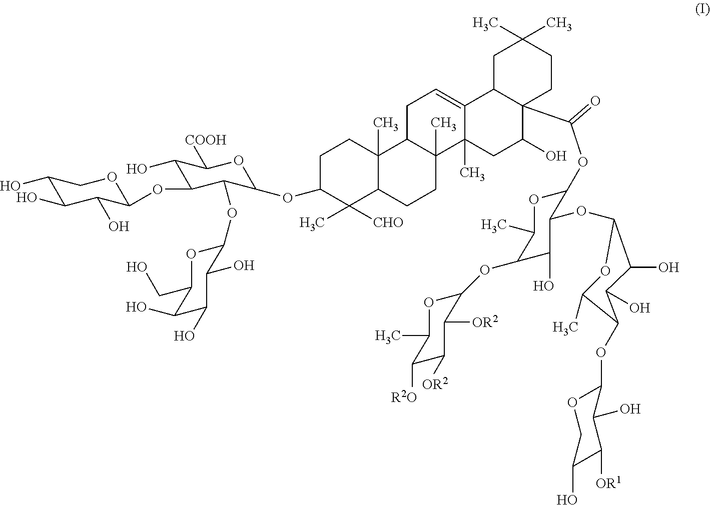

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin; and
(ii) incubating the cell with the nucleic acid in the presence of the saponin,

wherein the nucleic acid encoding for the CRISPR/Cas construct at least comprises a first part of the nucleic acid that encodes for a Cas and wherein the nucleic acid optionally comprises a second part of the nucleic acid that encodes for a gRNA or comprises a gRNA or is a gRNA.

Advantageously, a method is provided characterized in that the nucleic acid is a plasmid DNA.

Preferred is the method according to the invention, characterized in that the nucleic acid forms part of a nanoparticle which nanoparticle further comprises a nanoparticle-forming compound.

Particularly preferred is the method according to the invention, characterized in that the nanoparticle comprises the nanoparticle-forming compound poly-lysine peptide, wherein preferably the poly-lysine peptide consists of 5-25 lysine residues, more preferably the poly-lysine peptide consists of 16 lysine residues. Such as preferred poly-lysine peptide is also referred to as K₁₆ peptide.

It is part of the invention that the method or the use of the invention is characterized in that the nanoparticle comprises the nanoparticle-forming compound, such as a peptide, such as poly-lysine, and the nucleic acid in a mass ratio in a range of 3:1 to 15:1, preferably in a range of 3:1 to 9:1. Typically, the nanoparticle-forming compound is a poly-lysine peptide such as K₁₆ peptide. Preferred is the method wherein the nucleic acid is a plasmid DNA, at least encoding for a Cas, preferably Cas9 (SEQ ID NO: 1), and optionally also encoding for the gRNA, and wherein the nanoparticles comprise in addition to the plasmid DNA at least one poly-lysine peptide, preferably poly-lysine K₁₆ peptide (poly-lysine peptide consisting of sixteen lysine residues). Alternatively, the poly-lysine peptide is a peptide consisting of 8-25 lysine residues, preferably 10-20, more preferably 12-18. High in vitro transfection efficiency is obtained with the method of the invention when nanoparticles encompassing plasmid DNA, K₁₆ peptide are prepared and applied, and are contacted with cells in the presence of a saponin selected from GE1741 and SO1861, and preferably the saponin is GE1741. Typically, the saponin concentration during transfection of the plasmid DNA into selected target cells in vitro is 1 μg/mL-5 μg/mL, preferably 2-4 μg/mL. The inventors established that the saponin is not exhibiting toxic activity towards the cells with which the nanoplexes are contacted in the presence of the saponin such as GE1741 or SO1861, when the saponin is applied onto the cells together with the nanoplexes, at a saponin concentration of 1 μg/mL-5 μg/mL. Cells are typically contacted with a composition comprising the nanoplexes and a separate composition comprising the saponin, preferably GE1741. Thus, typically, the nanoparticles comprise the poly-lysine peptide and the nucleic acid, for example at least a plasmid DNA encoding for a Cas, preferably Cas9 (SEQ ID NO: 1), or a Cas with at least 90% sequence identity with this Cas9, preferably 90-99.5% sequence identity, such as 92%, 93%, 94%, 95% or 95-99%.

Preferred is the method according to the invention, characterized in that the saponin concentration is 1 μg/mL-5 μg/mL in step (ii) of the method. Typically, the concentration of the saponin during transfection of cells is 1.5-4.5 μg/mL, such as 2, 2.5, 3, 3.5 or 4 μg/mL, or any concentration therein between. Such saponin concentrations do not inflict toxicity towards the cells that are selected for transfection of the nucleic acid, according to the use and the method of the invention. Such saponin concentrations, wherein the saponin preferably is GE1741 or SO1841, more preferably, the saponin is GE1741, are suitable for efficient transfection of the nucleic acid, preferably a plasmid DNA encoding at least for a Cas, preferably Cas9 (SEQ ID NO: 1). Alternatively, the encoded Cas is Cas12a (SEQ ID NO:2) or Cas13a (SEQ ID NO: 3).

The inventors established that relatively high transfection efficacy was obtained when cells were contacted in vitro with nanoplexes comprising plasmid DNA with a size of at least 5.5 kbp, for example with a size of 6.5 kbp-8.5 kbp, such as 7 kbp-8.3 kbp, and comprising poly-lysine peptide such as K₁₆ peptide, in the presence of saponin SO1861 or GE1741. GE1741 is preferred, since with the application of GE1741, even more improved transfection efficiency was achievable compared to SO1861, when efficiency was compared with the results obtained with transfections applying the ‘gold standard’ Lipofectamin3000 according to the recommendations of the manufacturer. Therefore, application of GE1741 in the method of the invention and for the use according to the invention is particularly preferred. The nanoplexes are first prepared by mixing the nucleic acid such as a plasmid DNA with a size of 5.5 kbp or larger such as 5.5-9.5 kbp, and the poly-lysine peptide, such as K₁₆ peptide, and after such nanoplexes of nucleic acid and poly-lysine are obtained, these nanoplexes are optionally first further mixed with the saponin such as SO1861 or GE1741, before the nanoplexes consisting of the nucleic acid and the poly-lysine peptide are applied to cells in the presence of the saponin. Alternatively, the nanoparticles and the saponin are added to a cell culture separately such that the cells are transfected in the presence of the saponin, for example in the cell culture medium.

An embodiment is the method according to the invention, characterized in that the saponin is GE1741 according to formula (II):

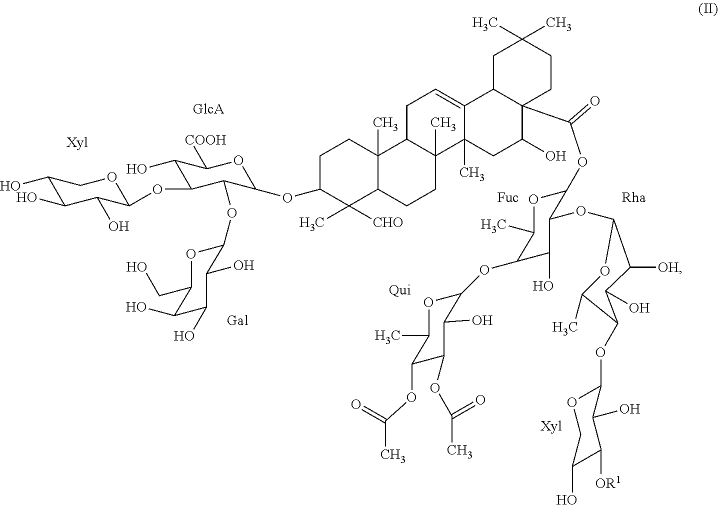

wherein R¹ is xylose;
or in that the saponin is SO1861 according to formula (III):

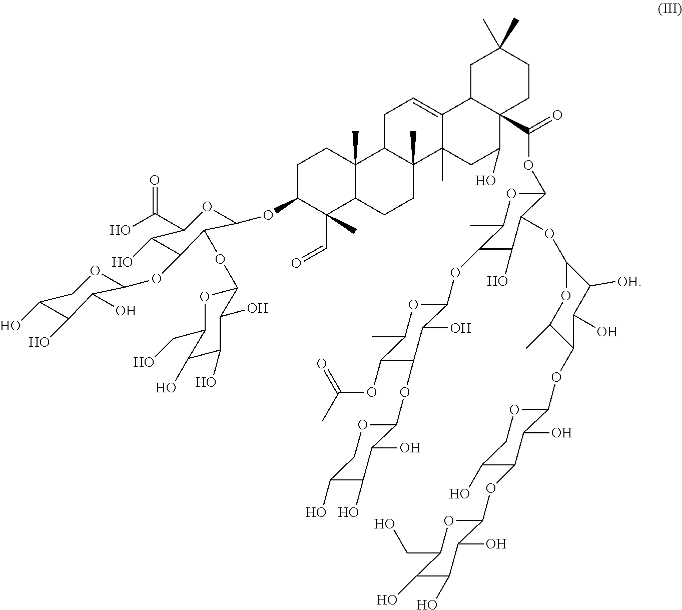

Preferred is the method of the invention or the use according to the invention, wherein the saponin is GE1741. Since efficient transfection is achieved with nanoplexes that are combined with GE1741 or SO1861 during transfection of target cells, saponins with a similar molecular structure according to the embodiments herein described, are also suitable for improving nucleic acid transfection efficiency, according to the use of the invention or according to the method of the invention.

Typically, in the method of the invention or according to the use of the invention, the saponin is applied at a concentration, during transfection of target cells, of 1-5 μg/ml, since at such concentration the saponin is non-toxic towards the cell, while transfection efficacy is still optimal when the positive influence of the presence of the saponin on the transfection efficiency of the nucleic acid comprised by the nanoplexes (nanoparticles) of the invention is considered. Preferably, the nanoplexes comprise the poly-lysine peptide K₁₆. Of course, shorter and longer poly-lysine peptides are equally suitable, as long as cell toxicity is not induced under influence of the alternative length of the poly-lysine peptide, and as long as transfection efficiency is not hampered by applying a poly-lysine peptide with a different length than K₁₆, for which transfection efficiency is at least 15%, preferably at least 20%, more preferably at least 25%, most preferably at least 30%. Typically, for transfecting plasmid DNA of a size of at least 5.5 kbp such as 5.5-10 kbp, or 6.5-9.0 kbp, application of K₁₆ peptide is highly beneficial when transfection efficiency is considered. In the nanoplexes applied in the method of the invention or applied for the use according to the invention, therefore, presence of K₁₆ peptide is preferred, although poly-lysine peptides with a shorter or longer length are also suitable, such as K₁₀-K₂₀.

Advantageously, in the method of the invention, the nucleic acid will be a nucleic acid encoding for the CRISPR/Cas construct that at least comprises a first part that encodes for a Cas and wherein the nucleic acid optionally comprises a second part that encodes for a gRNA or comprises a gRNA or is a gRNA. Particularly preferred is the use of the invention or the method according to the invention, characterized in that the nucleic acid encoding for the CRISPR/Cas construct is provided as part of a plasmid DNA, preferably in that at least the part of the nucleic acid encoding for the Cas part of the CRISPR/Cas construct is provided as part of a plasmid DNA, more preferably in that the plasmid DNA is for expressing the Cas and the gRNA of the CRISPR/Cas construct. Preferably, the plasmid DNA is encoding for Cas9 (SEQ ID NO:9) or for a Cas which has at least 90% sequence identity with this Cas9 protein. Typically, the plasmid DNA has a size of at least 6.0 kbp, such as 6.5-8.8 kbp. Typically, when the plasmid DNA encompasses the nucleic acid encoding for the Cas, preferably Cas9, and also encompasses the nucleic acid encoding for the gRNA, the size of the plasmid DNA is larger than 7.0 kbp, such as 7.5-9.5 kbp, or 8-8.5 kbp. It is beneficial to apply plasmid DNA that comprises the nucleic acid encoding for Cas, for example Cas9 (SEQ ID NO: 1), and the nucleic acid encoding for the gRNA, since only a single plasmid DNA has to be involved in the nanoplex formation with the poly-lysine peptide, preferably K₁₆ peptide, and subsequently in the formation of the mixture with the saponin (either before adding the nanoparticles to cells, or by co-adding the separate composition comprising nanoparticles and the separate composition comprising saponin to the cells), such as SO1861 or GE1741, preferably GE1741. Consistent batch-to-batch production is made more convenient and easy when only a single plasmid DNA has to be nanoplexed with the peptide (and optionally pre-mixed with the saponin, before being added to cells selected for transfection by the nanoplexed nucleic acid). However, the method and use of the invention are also suitable and applicable for application of a first plasmid DNA encoding forthe Cas, and a second plasmid DNA encoding forthe gRNA. In embodiments, the method and use of the invention are applied with a plasmid DNA encoding for Cas9 and green fluorescent protein (GFP).

Preferred is the use or the method of the invention, characterized in that the plasmid DNA comprises more than 6 kbp, preferably at least 7 kbp, more preferably at least 7.5 kbp, most preferably at least 8 kbp. For example, a plasmid DNA comprising a nucleic acid encoding for Cas9 is typically 6.5-7.5 kbp, and a plasmid DNA comprising a nucleic acid encoding for Cas9-GFP construct is typically 7.5-8.5 kbp, Such plasmid DNA sizes can be transfected efficiently when nanoplexes are prepared with poly-lysine peptide such as K₁₆ peptide, and optionally subsequently mixed with saponin, such as GE1741, which nanoplexes are contacted with the cells selected for transfection in the presence of the (pre-mixed) saponin. The inventors previously revealed in a general sense that toxic effects became apparent when cells were contacted with nanoplexes of a 16:1 mass ratio (16 parts peptide, 1 part nucleic acid, based on the total weight of the nanoplex (nanoparticle)). In addition, the inventors previously revealed in a general sense that an inefficient nucleic acid delivery became apparent when cells were contacted with nanoplexes of a 2:1 mass ratio (peptide:nucleic acid). Therefore, in view of absence of toxicity and in view of apparent nucleic acid delivery efficiency, nanoplexes are formulated in a mass ratio of 3:1 to 12:1 of peptide mass to nucleic acid mass, based on the total weight of the nanoplex (nanoparticle). Preferably, the mass ratio of nucleic acid, such as total peptide to total plasmid DNA, is 4:1 to 8:1, based on the total weight of the nanoplex, such as 4:1, 5:1, 6:1, 7:1 or 8:1. Transfection efficiency is typically at least 20%, under influence of the saponin such as SO1861 or GE1741, preferably GE1741, in the nanoplexes (nanoparticles) comprising the poly-lysine peptide such as K₁₄-K₁₉ peptide, preferably K₁₆ peptide, and comprising a single first plasmid DNA comprising the nucleic acid for encoding a Cas such as Cas9 (SEQ ID NO:1) and comprising the nucleic acid encoding for the gRNA, or comprising a first plasmid DNA encoding for said Cas and a second plasmid DNA encoding for said gRNA.

Preferred is the use of the invention or the method according to the invention, characterized in that the Cas encoding nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b). Preferably, the Cas encoding nucleic acid is part of a plasmid DNA, for example a plasmid DNA with a size of 6-9 kbp, typically 6.5-8.5 kbp. For Cas9, the gRNA encodes for the tracrRNA and the CRISPR RNA (crRNA), whereas when the nucleic acid encodes for Cas12a (SEQ ID NO:2) or for Cas13a (SEQ ID NO:3), the nucleic acid encoding for the gRNA, only encodes for the crRNA. When the nucleic acid that is applied in the method of the invention or is involved in the use of the invention, is encoding for Cas9 or a Cas with at least 90% sequence identity with the Cas9 according to SEQ ID NO:1, the encoded gRNA comprises CRISPR RNA (crRNA) and comprises trans-activating CRISPR RNA (tracrRNA), wherein the gRNA is a single-guide RNA (sgRNA) or a two-piece RNA, wherein the tracrRNA comprises a binding site for the Cas9.

A third aspect of the invention relates to a kit of parts for delivering a nucleic acid comprising more than 5.5 kilo base-pairs (kbp) into a cell in vitro comprising:
a first combination comprising or consisting of:
(a) a first container comprising a first plasmid DNA;
(b) optionally a second container comprising a second plasmid DNA;
(c) a third container comprising poly-lysine K₁₆ peptide;
(d) a fourth container comprising at least one saponin being a bisdesmosidic triterpenoid 12,13-dehydrooleanane-type saponin, preferably being one saponin, most preferably selected of: GE1741 and SO1861;
(e) instructions for use,

wherein the use at least comprises the preparation of nanoplexes of the poly-lysine K₁₆ peptide and the first plasmid DNA, and optionally the saponin,

wherein the first plasmid has a size of at least 5.5 kbp,

or
a second combination comprising or consisting of:
(a) a first container comprising a third plasmid DNA;
(b) a second container comprising poly-lysine K₁₆ peptide;
(c) a third container comprising at least one saponin being a bisdesmosidic triterpenoid 12,13-dehydrooleanane-type saponin, preferably being one saponin, most preferably selected of: GE1741 and SO1861;
(d) instructions for use,

wherein the use at least comprises the preparation of nanoplexes of the poly-lysine K₁₆ peptide and the third plasmid DNA, and optionally the saponin, wherein the third plasmid has a size of at least 5.5 kbp, or
a third combination comprising or consisting of:
(a) a first container comprising the first plasmid DNA;
(b) optionally a second container comprising a RNA oligonucleotide;
(c) a third container comprising poly-lysine K₁₆ peptide;
(d) a fourth container comprising at least one saponin saponin being a bisdesmosidic triterpenoid 12,13-dehydrooleanane-type saponin, preferably being one saponin, most preferably selected of: GE1741 and SO1861;
(e) instructions for use,

wherein the use at least comprises the preparation of nanoplexes of the poly-lysine K₁₆ peptide and the first plasmid DNA, and optionally the saponin, wherein the first plasmid has a size of at least 5.5 kbp, preferably wherein the first plasmid DNA is encoding for Cas9, and the second plasmid DNA is encoding for gRNA and/or the RNA oligonucleotide is a gRNA RNA oligonucleotide, or wherein the third plasmid DNA is encoding for Cas9 and encoding for gRNA.

Alternatively, the third aspect of the invention can relate to a kit of parts for delivering a nucleic acid encoding for a CRISPR/Cas construct into a cell in vitro comprising:
a first combination comprising or consisting of:
(a) a first container comprising a first plasmid DNA encoding for Cas9;
(b) optionally a second container comprising a second plasmid DNA encoding for gRNA;
(c) a third container comprising poly-lysine K₁₆ peptide;
(d) a fourth container comprising at least one saponin, preferably one saponin, of: GE1741 and SO1861;
(e) instructions for use,
wherein the use at least comprises the preparation of nanoplexes of the poly-lysine K₁₆ peptide and the first plasmid DNA, and optionally the saponin,
wherein the first plasmid has a size of at least 5.5 kbp,
or
a second combination comprising or consisting of:
(a) a first container comprising a third plasmid DNA encoding for Cas9 and encoding for gRNA;
(b) a second container comprising poly-lysine K₁₆ peptide;
(c) a third container comprising at least one saponin, preferably one saponin, of: GE1741 and SO1861;
(d) instructions for use,
wherein the use at least comprises the preparation of nanoplexes of the poly-lysine K₁₆ peptide and the third plasmid DNA, and optionally the saponin
wherein the third plasmid has a size of at least 5.5 kbp,
or
a third combination comprising or consisting of:
(a) a first container comprising the first plasmid DNA encoding for Cas9;
(b) optionally a second container comprising a gRNA RNA oligonucleotide;
(c) a third container comprising poly-lysine K₁₆ peptide;
(d) a fourth container comprising at least one saponin, preferably one saponin, of: GE1741 and SO1861;
(e) instructions for use,
wherein the use at least comprises the preparation of nanoplexes of the poly-lysine K₁₆ peptide and the first plasmid DNA, and optionally the saponin.
wherein the first plasmid has a size of at least 5.5 kbp.

The kit optionally comprises at least one further container containing a diluent or solvent, for diluting or dissolving the component contained by any one or more of the first, second, third, and if present, fourth container of the first, second or third combination, and/or for diluting or dissolving the components of the nanoplexes comprising at least the plasmid DNA and the poly-lysine peptide consisting of sixteen lysine residues, and optionally the at least one saponin, preferably either GE1741 or SO1861, more preferably GE1741. Preferably, the kit contains a container or vial comprising GE1741 as the sole saponin. Typically, the plasmid DNA has a size of 5.5 kbp-10 kbp, such as 6.5 kbp-7.5 kbp, typical for the first or third combination, or 7.5 kbp-8.5 kbp, typical for the second combination. The plasmid DNA preferably comprises a part of the DNA that encodes for Cas9 according to the amino acid sequence of SEQ ID NO: 1. Alternatively, the kit comprises plasmid DNA encoding for a Cas which has at least 90% identity with the amino acid sequence of Cas9 (SEQ ID NO: 1, see for example: www.uniprot.org/uniprot/Q99ZW2).

The encoded non-coding guide RNA of the first or second combination of the kit or the gRNA comprised by the third combination of the kit, comprises CRISPR RNA (crRNA) and comprises trans-activating CRISPR RNA (tracrRNA), wherein the guide RNA is a single-guide RNA or a two-piece RNA, wherein the tracrRNA comprises a binding site for the Cas9 encoded by the first or third plasmid DNA.

The use and/or method and/or kit of the invention is in particular suitable for obtaining a cell with a knocked-in gene in vitro, preferably a stably transfected knock-in cell in vitro. Preferably, the nucleic acid encoding for the protein, to be knocked-in in the target cell genome, is a DNA of the type of linear donor sequence (LDS), although also a circular DNA donor template is applicable for knocking-in the encoding nucleic acid into the genome of the target cell, by applying the method of the invention, or according to the use of the invention, and with the use of the kit of the invention. The LDS or the circular DNA donor template, for example in the form of a vector or plasmid DNA, is co-transfected with the nucleic acid encoding for the Cas, preferably Cas9 (SEQ ID NO: 1), preferably as part of a plasmid DNA, which plasmid DNA preferably also comprises a nucleic acid encoding for the gRNA, although for the co-transfection, the gRNA can also be provided as an RNA or as a further plasmid DNA encoding for the gRNA. According to the invention, the nucleic acid(s) encoding for the Cas and the gRNA, preferably the one or two plasmid DNAs, more preferably, a single plasmid DNA, is provided as a nanoparticle (nanoplexed components, forming a nanoparticle) comprising or consisting of, preferably comprising the plasmid DNA(s), a poly-lysine peptide, preferably K₁₆ peptide, and optionally a saponin selected from SO1861 and GE1741, preferably the saponin is GE1741. Preferred are nanoparticles comprising the nucleic acid and the poly-lysine peptide, which nanoparticles are mixed with the saponin during the transfection of target cells with the nucleic acid. Cells selected for knocking-in upon (stable) transfection of the linear or circular DNA donor template, preferably an LDS, are contacted with the saponin and the nanoparticles and the DNA donor template, such that the nucleic acid encoding for the Cas, preferably Cas9 (SEQ ID NO: 1), the nucleic acid encoding for the gRNA, and the DNA donor template, preferably an LDS, are transfected into the cell in vitro, and such that subsequently the donor DNA is knocked-in the target position of the cell's genome. Preferably, the use of the invention, the method of the invention, the application of the kit of the invention and/or the application of the nanoparticles of the invention in combination with the saponin, result in efficient transfection of a target cell, wherein ‘efficient’ refers to a transfection rate of at least 20%, such as at least 25% or at least 30% or at least 35%, and preferably the knocking-in of a selected nucleic acid into the cell's genome is a stable transfection, that is to say, the knocked-in nucleic acid remains in the cell's genome after a single or more passages of the cell.

A fourth aspect of the invention relates to a composition comprising a nanoparticle, suitable for in vitro delivery of a nucleic acid into a cell, wherein the nanoparticle comprises or consists of:
(i) the nucleic acid comprising more than 5.5 kilo base-pairs (kbp);
(ii) a poly-lysine peptide, and
(iii) a saponin being a bisdesmosidic triterpenoid 12,13-dehydrooleanane-type saponin.

A particular embodiment of the fourth aspect of the invention, can advantageously relate to a composition comprising a nanoparticle, suitable for in vitro delivery of a nucleic acid encoding for a CRISPR/Cas construct into a cell, wherein the nanoparticle comprises or consists of:
(i) the nucleic acid encoding for a CRISPR/Cas construct;
(ii) a poly-lysine peptide,

wherein the nucleic acid encoding for a CRISPR/Cas construct at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA.

Preferred is the composition of the invention, comprising a saponin according to formula (I):

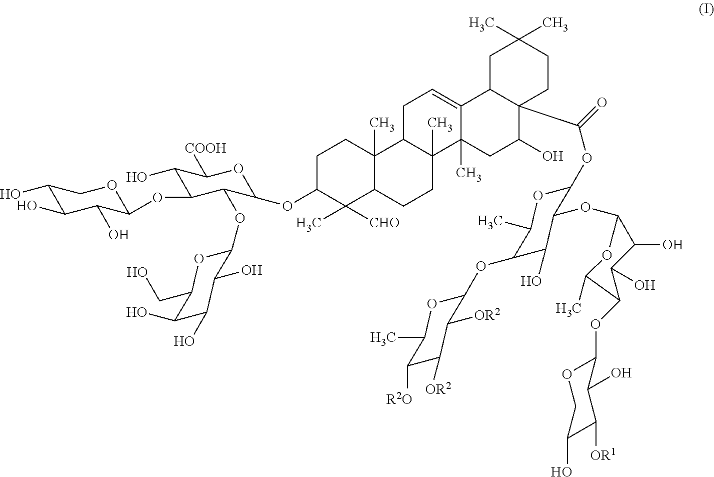

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin.

According to the invention, the composition can be first mixed with the saponin, before the composition is applied onto cells that are selected for transfection with the nucleic acid of choice that comprises more than 5.5 kbp, for example a CRISPR/Cas construct, or the composition is contacted with the cells and saponin is also added to the cells, by applying a first composition comprising the saponin to the cells and by applying the composition of the invention to the cells.

An embodiment is the composition according to the invention, comprising the saponin, wherein R¹ of the saponin according to formula (I) is a xylose residue and/or in that the saponin carries exactly two acetyl groups.

An embodiment is the composition according to the invention, comprising the saponin, wherein the acetyl groups of the saponin according to formula (I) are bonded to the oxygen atoms in C3 position and in C4 position of the corresponding quinovose residue of the saponin.

An embodiment is the composition according to the invention, comprising the saponin, wherein one of the R² residues of the saponin according to formula (I) is a xylose residue, one of the R² residues is an acetyl group and one of the R² residues is H.

An embodiment is the composition according to the invention, comprising the saponin, wherein the xylose residue of the saponin according to formula (I) is bonded to the oxygen atom in C3 position of the corresponding quinovose residue and in that the acetyl group is bonded to the oxygen atom in C4 position of the corresponding quinovose residue of the saponin.

An embodiment is the composition according to the invention, comprising the saponin, wherein R¹ of the saponin according to formula (I) is a xylose residue and in that two R² groups of the saponin according to formula (I) are acetyl groups which are bonded to the oxygen atoms in C3 position and in C4 position of the corresponding quinovose residue, and in that the third R² group is H.

An embodiment is the composition according to the invention, comprising the saponin, wherein the saponin is GE1741 according to formula (II):

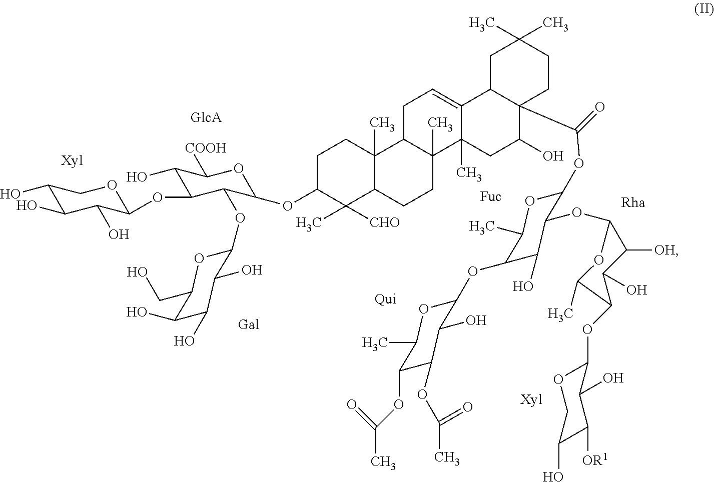

wherein R¹ is xylose;
or wherein the saponin is SO1861 according to formula (III):

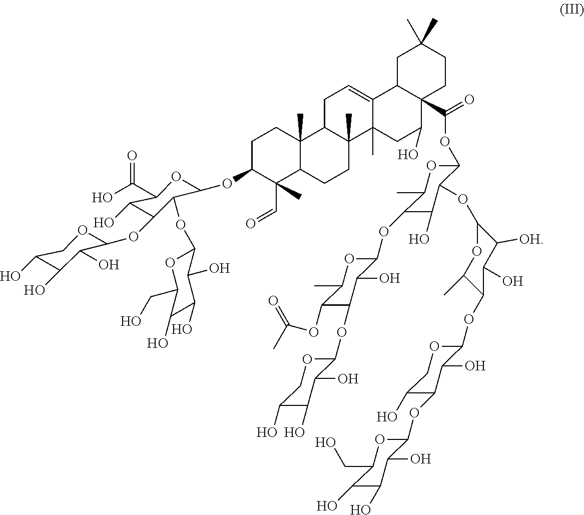

Preferred is the composition according to the invention, wherein the poly-lysine peptide consists of 5-25 lysine residues, more preferably the poly-lysine peptide consists of 16 lysine residues.

Typically, in the composition according to the invention, the nanoparticle comprises the poly-lysine peptide and the nucleic acid in a mass ratio in a range of 3:1 to 15:1, preferably in a range of 3:1 to 9:1. The composition either or not comprises the saponin. Typically, the composition of the invention does not comprise the saponin, and when the composition is applied for transfecting a cell with the construct, the cells are co-incubated with the composition of the invention and with the saponin of the here above outlined embodiments.

Preferred is the composition according to the invention, wherein the nucleic acid is part of a plasmid DNA. As explained above, in certain embodiments, preferred is the composition wherein the nucleic acid at least comprises a first part of the nucleic acid that encodes for a Cas and wherein the nucleic acid optionally comprises a second part of the nucleic acid that encodes for a gRNA or comprises a gRNA or is a gRNA.

Particularly preferred is the composition of the invention, wherein the plasmid DNA comprises more than 6 kbp, preferably at least 7 kbp, more preferably at least 7.5 kbp, most preferably at least 8 kbp.

Preferred is the composition according to the invention, wherein the Cas encoding first part of the nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b).

A fifth aspect of the invention relates to a nanoparticle suitable for in vitro delivery of a nucleic acid into a cell, the nanoparticle comprising or consisting of:
(i) a nucleic acid encoding for a CRISPR/Cas construct;
(ii) a poly-lysine peptide; and
(iii) a saponin according to formula (I):

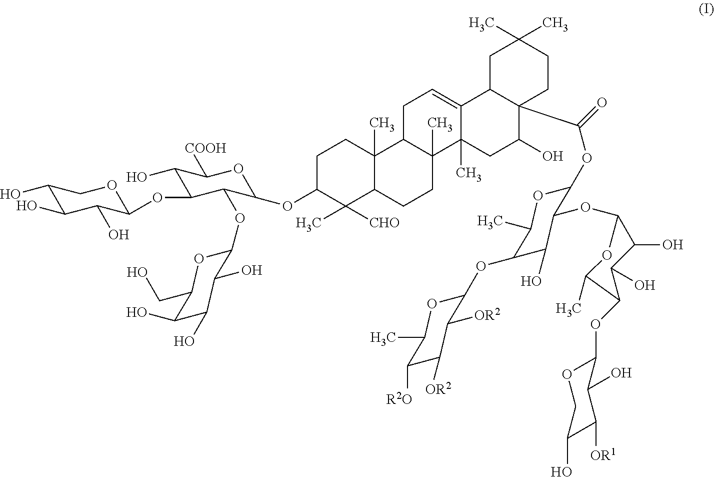

wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin, wherein the nucleic acid encoding for a CRISPR/Cas construct at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA.

Embodiments of the invention are the nanoparticle according to the invention, wherein R¹ of the saponin according to formula (I) is a xylose residue and/or in that the saponin carries exactly two acetyl groups.

Suitable is the nanoparticle according to the invention, wherein the acetyl groups of the saponin according to formula (I) are bonded to the oxygen atoms in C3 position and in C4 position of the corresponding quinovose residue of the saponin.

Also suitable is the nanoparticle according to the invention, wherein one of the R² residues of the saponin according to formula (I) is a xylose residue, one of the R² residues is an acetyl group and one of the R² residues is H.

In certain embodiments, the nanoparticle according to the invention comprises the saponin of formula (I), wherein the xylose residue of the saponin according to formula (I) is bonded to the oxygen atom in C3 position of the corresponding quinovose residue and in that the acetyl group is bonded to the oxygen atom in C4 position of the corresponding quinovose residue of the saponin.

In certain embodiments, the nanoparticle according to the invention comprises the saponin of formula (I), wherein R¹ of the saponin according to formula (I) is a xylose residue and in that two R² groups of the saponin according to formula (I) are acetyl groups which are bonded to the oxygen atoms in C3 position and in C4 position of the corresponding quinovose residue, and in that the third R² group is H.

Preferred is the nanoparticle according to the invention, wherein the saponin is GE1741 according to formula (II):

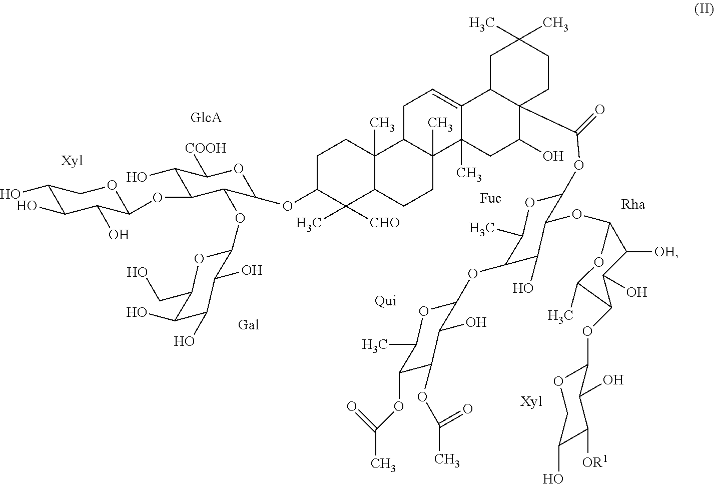

wherein R¹ is xylose;
or wherein the saponin is SO1861 according to formula (III):

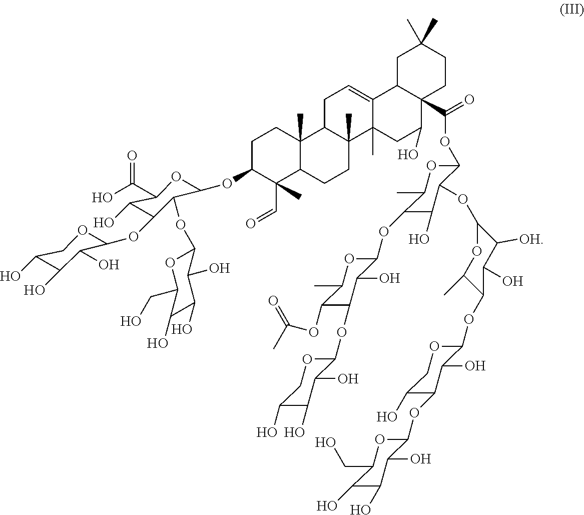

Preferably, the nanoparticle comprises SO1861 or GE1741, more preferably GE1741.

Preferably, for the nanoparticle according to the invention, the poly-lysine peptide consists of 5-25 lysine residues, more preferably the poly-lysine peptide consists of 16 lysine residues.

Typically, for the nanoparticle according to the invention, the nanoparticle comprises the nucleic acid and the poly-lysine peptide in a mass ratio in a range of 3:1 to 15:1, preferably in a range of 3:1 to 9:1. Preferably, the range is selected from 4:1-8:1.

Preferred is the nanoparticle according to the invention, wherein the nucleic acid is part of a plasmid DNA. The plasmid DNA preferably comprises a first part of the nucleic acid encoding for a Cas, preferably for Cas9 (SEQ ID NO: 1), and a second part of the nucleic acid encoding for a gRNA.

Typically, for the nanoparticle according to the invention, the plasmid DNA comprises more than 5.5 kbp, preferably at least 6 kbp, more preferably at least 7 kbp, most preferably at least 7.5 kbp. The plasmid DNA comprises the nucleic acid encoding for a CRISPR/Cas construct. The nucleic acid encoding for a CRISPR/Cas construct at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA.

It is preferred that for the nanoparticle according to the invention, the Cas encoding first part of the nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b). More preferred, the Cas is the Cas9.

The composition of the invention comprising a nanoparticle, suitable for in vitro delivery of a nucleic acid encoding for a CRISPR/Cas construct into a cell, or the nanoparticle of the invention, are particularly suitable for application in the method according to the invention, for transfecting a cell with the nucleic acid in the presence of a saponin. The use of the invention is typically involving such a composition of the invention or such a nanoparticle of the invention, wherein the saponin is co-administrated to cells that are to be transfected, or wherein the saponin is part of the composition or part of the nanoparticle.

The invention is further illustrated by the following examples, which should not be interpreted as limiting the present invention in any way. Modifications and alternative implementations of some parts or compounds are possible, and are included in the scope of protection as defined in the appended claims.

### EXAMPLES

### Nanoplex Characterization

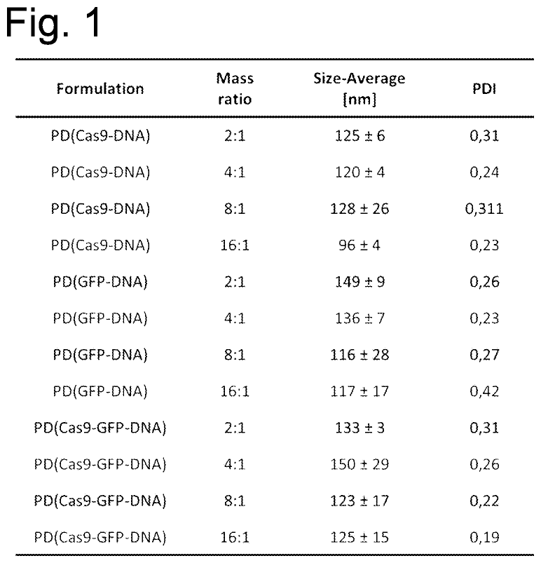

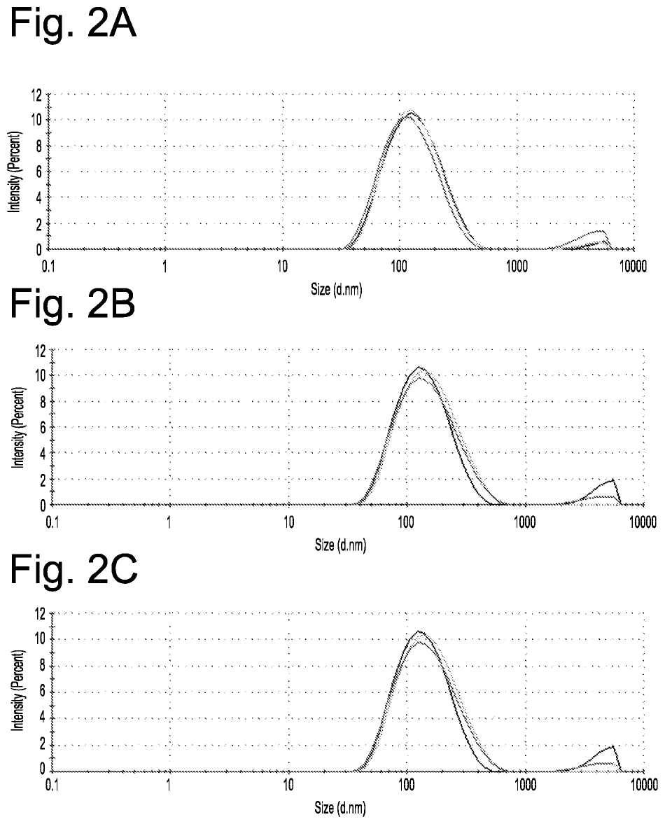

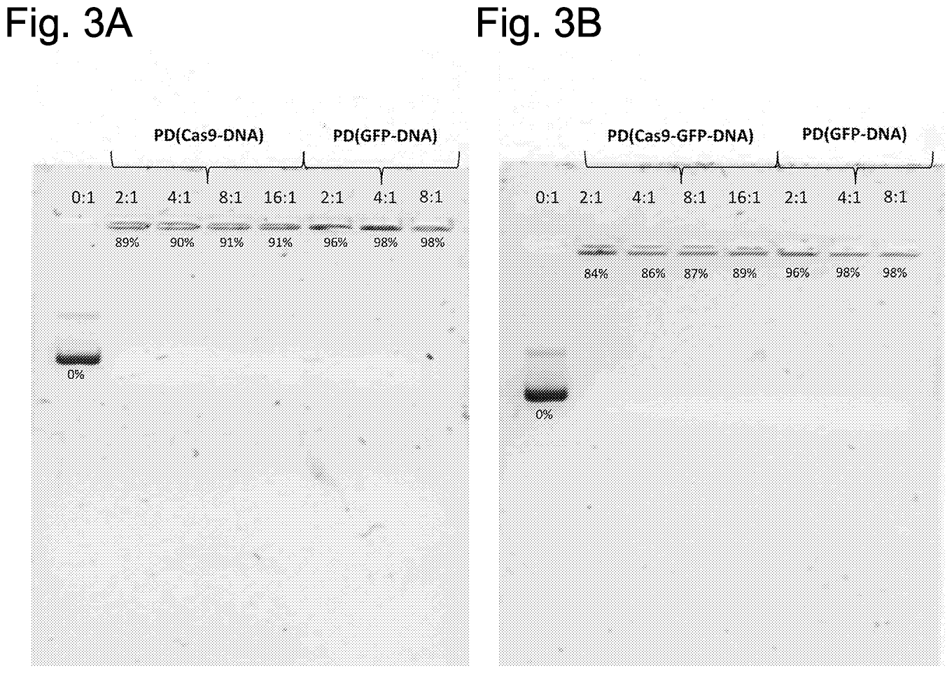

The inventors managed to perform the non-trivial complexation of the relatively large plasmid-DNA (8.216 bp) into oligo-lysine-based nanoplexes (nanoparticles). Such complexation had previously only achieved with plasmid in the range of 4-5 kbp (Sama et al. 2017). Nanoplexes, consisting of K₁₆-peptides and DNA-plasmids, so-called ‘PD-nanoplexes’, were formulated in different mass ratios (FIG. 1, FIG. 2) and the DNA-complexation was determined qualitatively (gel-retention assay) and quantitatively (fluorescence intensity measurement) (FIG. 3). The plasmid DNA encoded for Cas9. The Cas9 amino-acid sequence is provided in the list of sequences here below, as SEQ ID NO: 1. The Cas9 sequence is Cas9 from Streptococcus pyogenes serotype M1 and the sequence is retrieved from www.uniprot.org/uniprot/Q99ZW2, where it was listed as >sp|Q99ZW2|CAS9_STRP1 CRISPR-associated endonuclease Cas9/Csn1.

### Size & Size Distribution

The Cas9- and Cas9-GFP-DNA loaded PD-nanoplexes were complexed in different mass ratios. Size and PDI (polydispersity index) provide an initial assessment of the mass ratio for the optimal formulation in terms of stability and transfection efficiency (FIG. 1). The size values showed a tendency to smaller particles with higher mass ratios, when the mass ratio between the mass of applied peptide and the mass of applied nucleic acid is considered. The PDI indicated a mono-disperse particle suspension for all formulations, which was confirmed with the size distribution assessment, shown exemplary for the 4:1 mass ratio (peptide:nucleic acid) formulations (FIG. 2).

### Gel Retention

Without wishing to be bound by any theory, in the gel electrophoresis, dyes like ethidium bromide or SYBR Safe DNA Gel Stain have the purpose of making the double-stranded nucleic acids visible via fluorescence after intercalation. Is the DNA incorporated compactly into the nanoplexes, the nucleic acids do not run towards the anode after applying voltage. The DNA inside the nanoplexes remains in the gel pocket, because of the positively charged oligo-peptides a compensation of the negative charge is achieved.

DNA-plasmids, coding for Cas9, Cas9-GFP and—in comparison—GFP were therefore complexed in different mass ratios and transferred into the gel pockets. After finalizing the electrophoresis it was observed, that even with large plasmids (Cas9-ca. 7 kbp, Cas9-GFP-ca. 8 kbp) a DNA-complexation is possible and achieved (FIG. 3).

In order to quantify the complexation or determine the complexation efficiency, a fluorescence based quantification assay was conducted after nanoplex formulation. A dye (Quant-IT™ PicoGreen™, Thermo Fisher), fluorescing after binding to DNA, was used to calculate the amount offree DNA—complexed DNA cannot be accessed by the dye—and with that the complexation efficiency was calculated.

The experiment revealed that with increasing amount of peptide (poly-lysine K₁₆-peptide), a slightly increased efficiency was observed, which was efficient generally for all mass ratios (84-98%). In addition, with a larger plasmid size, a less efficient complexation was observed (FIG. 3).

The inventors previously revealed in a general sense that toxic effects became apparent when cells were contacted with nanoplexes of a 16:1 mass ratio. In addition, The inventors previously revealed in a general sense that an inefficient nucleic acid delivery became apparent when cells were contacted with nanoplexes of a 2:1 mass ratio due to the N/P ratio. Therefore, toxicity and nucleic acid delivery efficiency was assessed with PD-nanoplexes, formulated in a 4:1 and 8:1 ratio.

### Cell Transfection

### Cas9-GFP-DNA-Transfer

JIMT-1-cells (human breast carcinoma) and Neuro-2A-cells (murine neuroblastoma) were transfected with nanoplexes, containing Cas9-GFP-plasmid, with and without saponin GE1741-co-application or saponin SO1861-co-application (that is to say, the saponin is added to the cell culture together with the addition of the nanoparticles comprising the nucleic acid that is to be transfected in the presence of the saponin), in a non-toxic concentration when the saponins are considered. The mass ratios for the peptide and the nucleic acid (plasmid DNA) in the nanoparticles were selected considering the dynamic light scattering (DLS) measurements and considering the aforementioned earlier assessed toxicity and earlier performed efficiency studies. The transfection efficiency of all transfection conditions was measured after 48 h incubation time via flow cytometry by comparison to the negative control in terms of fluorescence intensity (FIG. 4). As comparison, the efficiency of saponin-based-transfection was compared with the commercially available transfection enhancers Lipofectamine3000™. The trademark Lipofectamin™ is being seen as gold standard in the field of non-viral transfections. The determination of transfection efficiency was possible with the GFP (green fluorescing protein)-gene, which was co-transfected as a Cas9-GFP-fusion protein gene, in the presence or absence of saponin during contacting cells with the nucleic acid. A successful transfection of the DNA plasmid comprising Cas9-GFP, and therefore indirect delivery of Cas9 protein upon intracellular transcription of the Cas9 DNA inside the successfully transfected cell, would be shown as green fluorescence caused by the co-transfected GFP DNA.

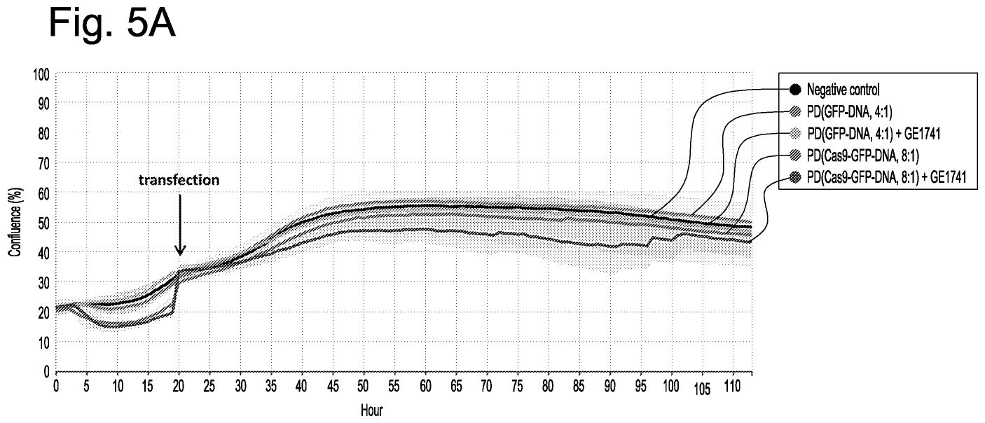

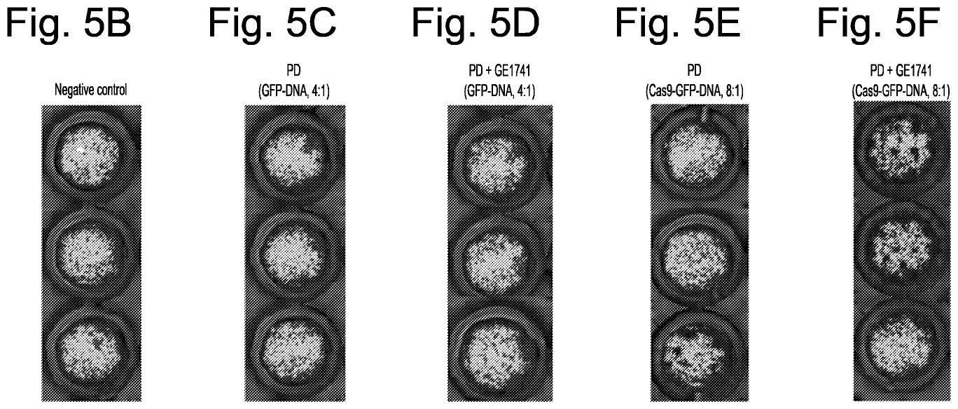

In the course of the transfection process of Neuro-2A-cells (seeding, transfection, incubation), the cells were fully imaged and analyzed for confluence by a live-cell imager (Cytosmart Omni). Via cloud-based algorithms, which processed the visual images, the Cytosmart provided a valuable measure of viability in terms of confluence. With this simultaneous experiment, no significant toxic effects were observed for the transfections with and without saponin-Co-application (FIG. 5).

The conducted tests provided the surprising insight that large DNA plasmids (with a size of at least 7 kbp) could be efficiently transfected in the cell lines when saponin was present during cell transfection, and the results of the conducted tests gain more impact, when taking account of the fewer amount of nucleic acid molecules, which are transfected, compared to relatively smaller GFP-plasmids with a size of smaller than 5 kbp. As the applied amount of nucleic acid remains 500 ng/well, but the size of the transfected DNA plasmid, and therefore the molecular mass of the plasmid, changes significantly, the efficiency is even higher than presented.

### Cas9-DNA-Transfer

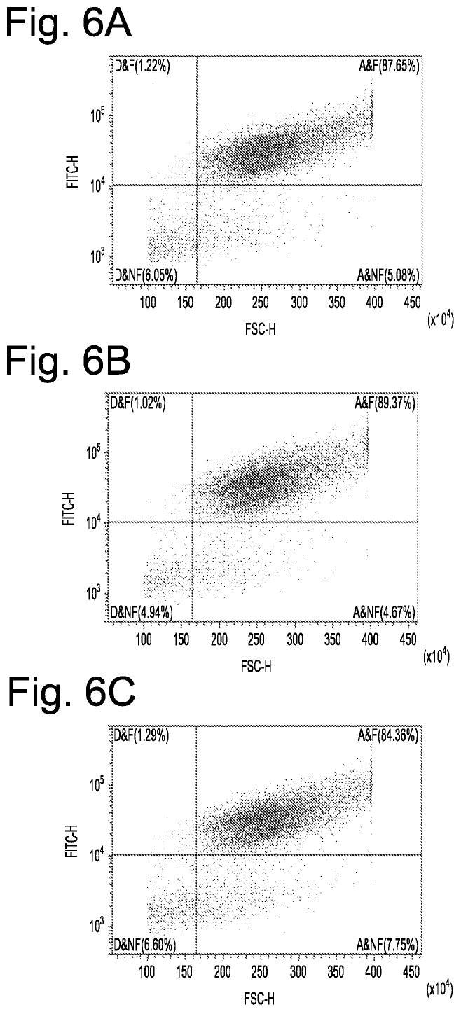

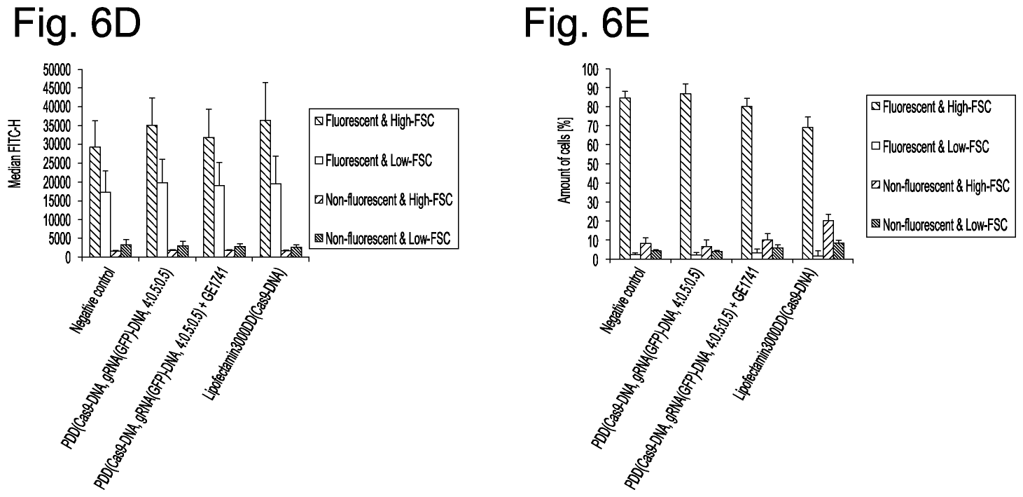

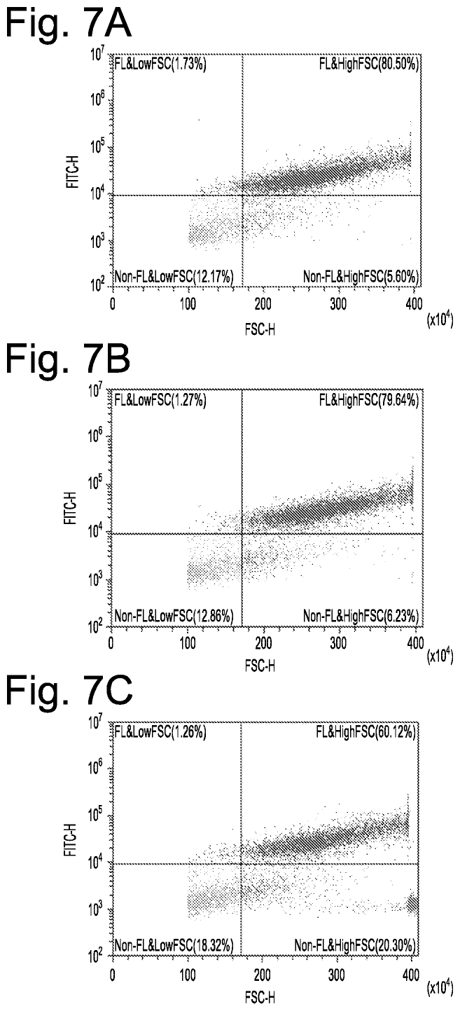

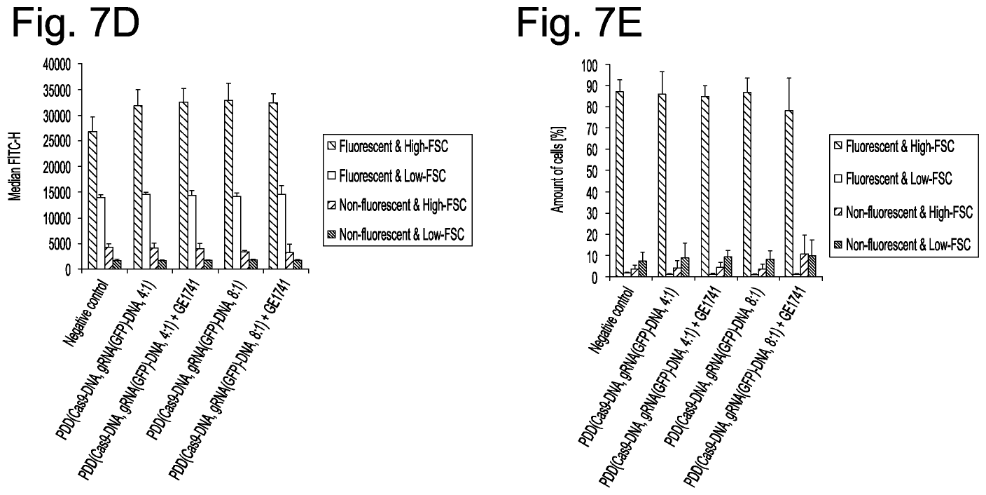

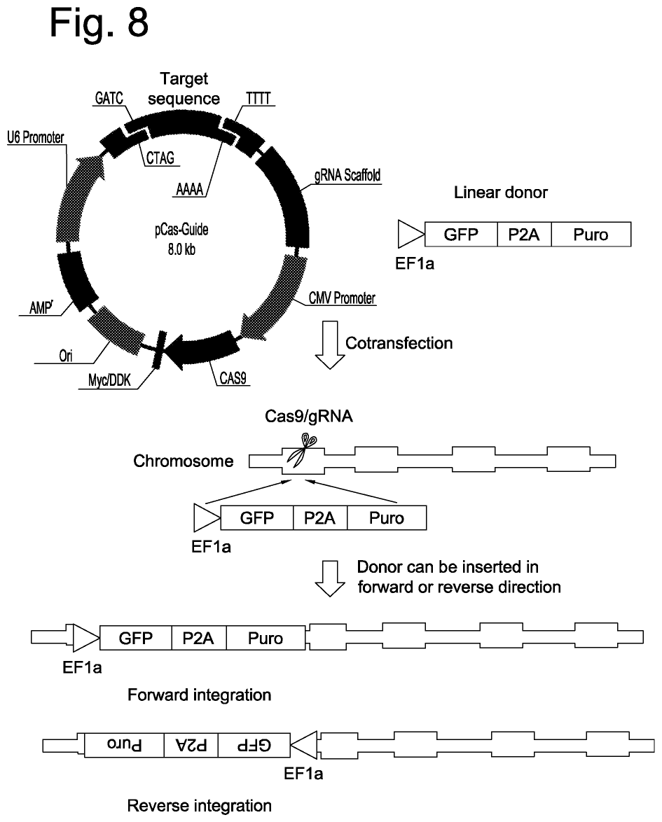

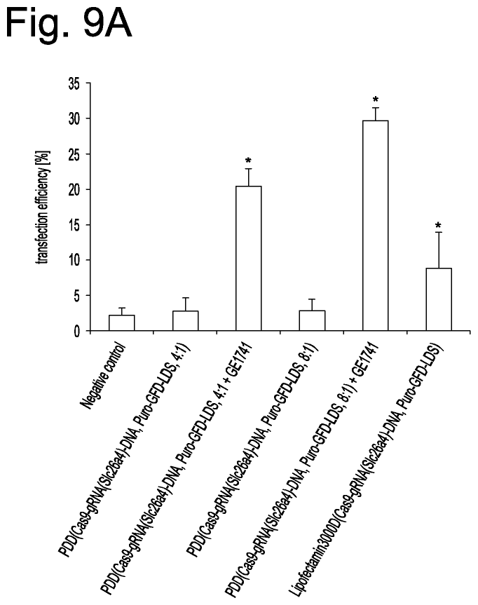

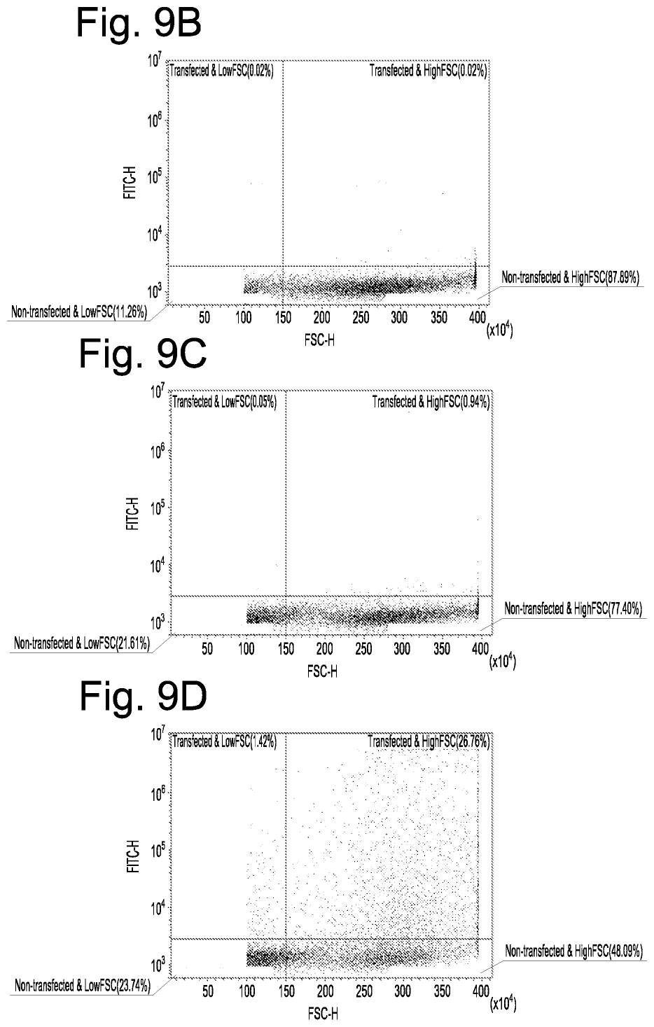

After the successful delivery of Cas9-DNA with GFP-driven fluorescence has been established, the inventors next tested the functionality of Cas9-transfections. GFP-expressing Neuro-2A-GFP-cells were therefore acquired. The aim of the test was to show and visualize the knock-out of the GFP-gene with a CRISPR-Cas9-gene knock-out. Therefore, Cas9-DNA (i.e. a plasmid DNA) was, either co-transfected with a gRNA-DNA-plasmid (PDD), or delivered in one plasmid DNA (“All-In-One-Plasmid”) coding for Cas9 and gRNA (PD) and expressing both Cas9 and guide RNA (FIG. 8, FIG. 9). The effect of GE1741-co-application during transfection of the cells was investigated via decrease of fluorescence intensity (FITC-H median & separation of the dot plot in FITC-H & FSC related quarters). Both experiments revealed a reduction of fluorescence (10% and 11% of all cells were not considered fluorescent, respectively) (FIG. 6, FIG. 7). Taking into account that 30% of all cells were successfully transfected with Cas9-DNA, the results showed efficient and significant gene knock-out to some extent. To improve gene knock-out efficiency, a number of different gRNA-sequences was tested, in order to select the most efficient target sequence for the gRNA. The initially and further applied gRNA-sequences were obtained through a database with efficiency information. For selection of an optimal gRNA sequence, the GFP-gene sequence of the transfected cell line has to be known as many GFP-sequences are available. Further, the gene knock-out requires a sensitive assay, by which minimal changes can be detected. Within promotor-driven highly fluorescing cells, single gene knock-out possibly could not be identified.

### Cas9-gRNA-DNA- & LDS-Transfer

The integration of a linear donor sequence (LDS, also referred to as a linear DNA donor template) into the genomic DNA right after strand break, is a method to knock-in and, with that, stably transfect a cell with a gene of interest. This way, with the CRISPR-Cas9 technique, a knock-out und knock-in can be established. LDS, coding for a reporter gene like GFP and resistance genes like Puromycin facilitate the detection of successful Cas9 modifications as well as the isolation of the respective successfully transfected cells. A first kit is provided by the inventors, comprising a first container containing an All-In-One plasmid (the first plasmid DNA), coding for Cas9 and gRNA, and a second container containing the LDS (a linear donor sequence DNA serving as a DNA donor template), coding for GFP and Puromycin, the All-In-One plasmid and the LDS selected for co-transfection into the cell (FIG. 8), the kit further comprising instructions for transfecting the first plasmid and the LDS into a target cell. Similarly, a second kit is provided by the inventors, comprising a container containing an All-In-One plasmid (the first plasmid), coding for Cas9 and gRNA, and containing the LDS, coding for GFP and Puromycin, the All-In-One plasmid and the LDS selected for co-transfection into the cell (FIG. 8), the kit further comprising instructions for transfecting the first plasmid and the LDS into a target cell. The first kit and the second kit may further comprise a vial containing poly-lysine K₁₆ peptide, for mixing with the first plasmid and optionally for mixing with the LDS, or for mixing with the combination of the first plasmid and the LDS, for providing nanoplexes of the peptide and the first plasmid and (optionally) the LDS. Alternatively, the first kit or the second kit comprises the first container further containing the poly-lysine K₁₆ peptide or comprises the container further containing the poly-lysine K₁₆ peptide, for providing nanoplexes of the peptide and the first plasmid and (optionally) the LDS. A GFP-mediated fluorescence proves the delivery of the LDS, which codes for the GFP, into the nucleus. The transfected cells, selected via Puromycin selection pressure, would express GFP-fluorescence over several passages as a proof for stable LDS-integration into the genome after Cas9-induced strand break.

The co-transfection of Neuro-2A-cells with the plasmid DNA encoding for the Cas9 and the gRNA, and the LDS encoding for the GFP and the Puromycin, was done in a poly-lysine K₁₆ peptide—nucleic acid ratio of 4:1 and 8:1, and plasmid DNA and LDS was complexed equally (500 ng/well). Transfections of cells were performed with and without GE1741 in the cell culture medium in which the cells were cultured. The transfection efficiency under influence of the absence or presence of the saponin was compared to transfection under influence of the presence of Lipofectamin3000 during transfection of the cells. The expression of fluorescence for over 30% of all transfected cells shows a successful delivery of LDS and the expression of GFP as a consequence, when the saponin was present during the transfection. Lipofectamin3000 did not reach comparable results (<10%) (FIG. 9).

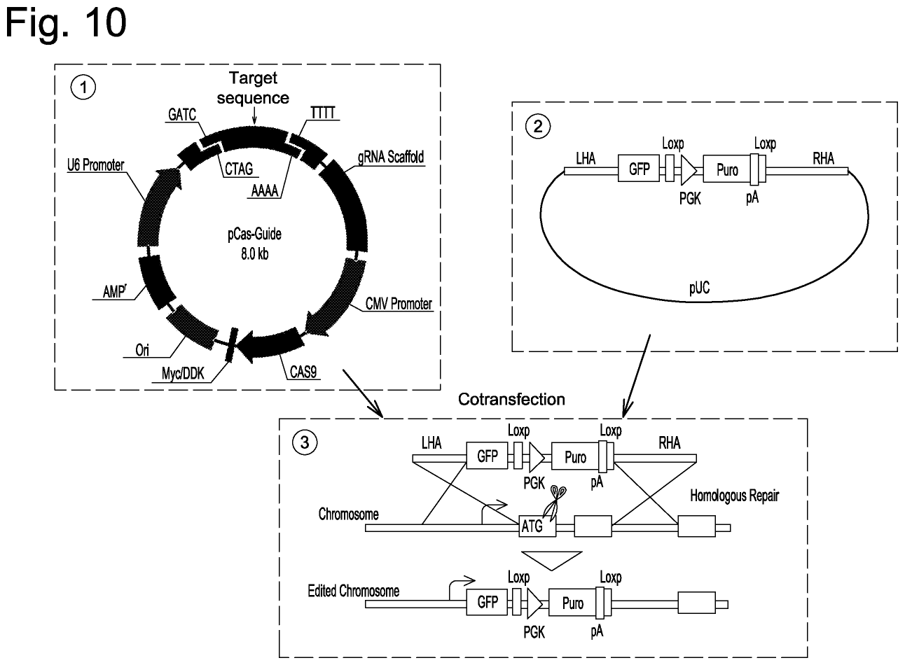

The integration of donor sequence into the genome can be performed via non-homologous recombination (‘NHR’) and homologous recombination (‘HR’). While the linear donor sequence is integrated forward and reverse (FIG. 8), a homologous integration (homologous recombination, ‘HR’) is only possible with homologous arms, resulting in a solely forward integration (FIG. 10). Both techniques may present advantages and disadvantages. Linear donor sequence (LDS) DNA can be integrated via NHR more easily into the strand break compared to integration applying HR, however, linearized nucleic acids are degraded relatively rapidly by DNAses. The more complex homologous integration of donor sequences is faced by the stability of its vector. Besides the tested Knock-out kit against the solute carrier 26a4 (FIG. 9), two other LDS-donor-kits (Slc19a3, Slc25a24) and two HR-based kits were tested (Slc4a4, Slc11a2). For each kit, two different gRNA-sequences were examined. The results show that no fluorescence expression could be detected for transfection, using homologous recombination (FIG. 11). Transfections with LDS, however, showed a significant efficiency increase, when the saponin was present during the transfection. The efficiency levels reached after the transfection with Slc26a4-target were the highest.

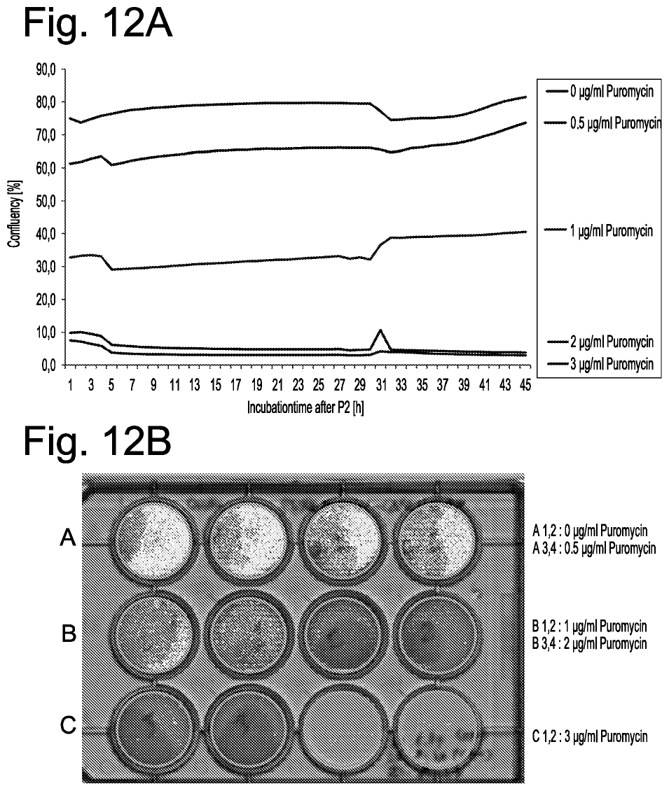

Subsequent to the co-transfection of Cas9-gRNA-Plasmid and LDS in the presence of GE1741, a Puromycin-mediated selection enabled the isolation of transfected cells and facilitates the identification of donor sequence integration into the genome. For this purpose, different Puromycin concentrations were applied to the transfected cells, in the presence of GE1741. The optimal puromycin concentration should optimally kill non-transfected/non-resistant cells, while sparing cells, which received the resistance through the donor sequence. While 0.5 μg/mL of Puromycin achieved no significant effect on the Neuro-2A-cells and 1 μg/mL merely an incomplete selection, 2-3 μg/mL diminished nearly all cells and was therefore qualified for further steps (FIG. 12).

The cells were allowed to grow by maintaining the Puromycin concentration. The genomic DNA was extracted, when confluence was reached. With designed primers (forward primer at Slc26a4 start, reverse primer at Slc26a4 end) the presence of the integrated donor cassette would show a targeted integration by CRISPR-Cas9. The fluorescence could be still seen five days after transfection.

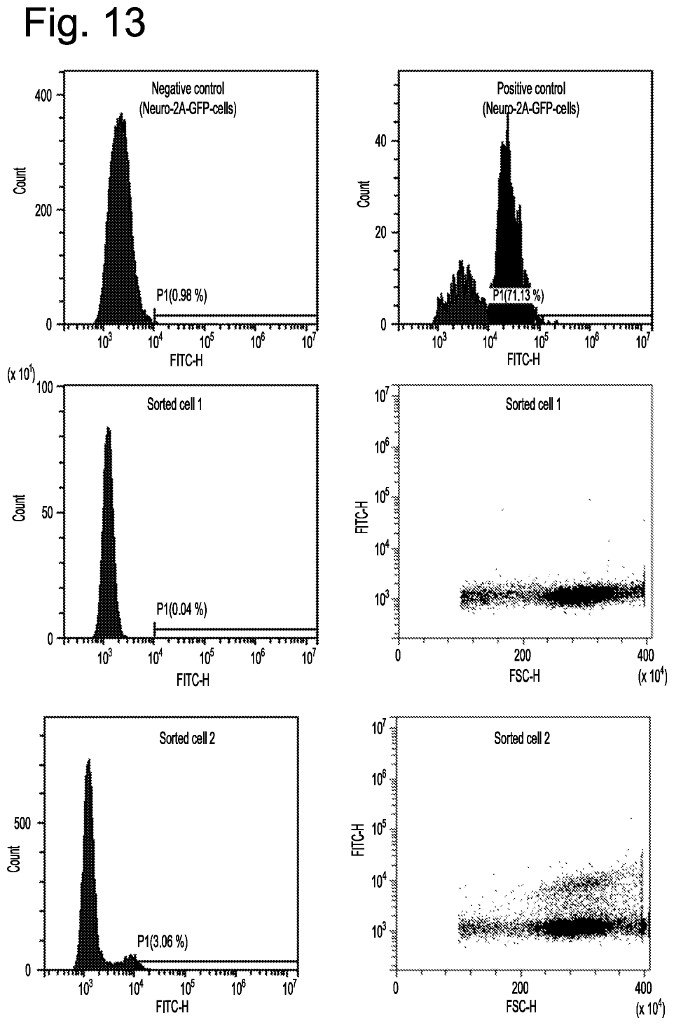

Another knock-in-transfection under influence of the presence of GE1741 was performed and the positive population was sorted and cultivated via fluorescence activated cell sorting (FACS). The positive population and new Neuro-2A-clones were further monitored for confluence and fluorescence. Fluorescence activated cell sorting (FACS) for isolation of modified cells was performed as alternative isolating method of genetically modified cells. Highly fluorescing Neuro-2A-cells were selected via FACS and isolated in a 96-well-plate and allowed to grow until confluence. The confluent wells were trypsinized and applied to flow cytometry. Untreated Neuro-2A-cells and untreated Neuro-2A-GFP-cells served as negative and positive control. While some cell clone population showed no fluorescence, others revealed two different populations (FIG. 13).

After detailed evaluation of the examples and tests, the following benefits achieved by applying the method of the invention and by using the nanoplexes in combination with a saponin, according to the method and use of the invention, or by applying the composition comprising the nanoparticles of the invention or the nanoplexes of the invention, including co-transfection with saponin, became apparent. An increased delivery of Cas9-DNA with the Co-application of triterpenoid saponins such as GE1741 or SO1861, preferably GE1741 is apparent. Formulation ratios and concentrations, which do show sufficiently low cell toxicity and which exhibit improved and efficient cell entry, are established by the inventors, when the mass ratio of poly-lysine to nucleic acid is considered: preferred is a ratio selected from 2:1-15:1, and preferred is a ratio selected from 4:1-8:1. The timing of application of the plasmid DNA (and the saponin) to the cells was sufficiently accurate for establishing gene knock-out and gene knock-in. In the examples, gRNA sequences were used which provide efficient gene knock-out. With sensitive assays, cells which are single knock-outs within a cell population, can be identified.

Thus, the surprisingly increased delivery of large constructs like Cas9-DNA (>8 kbp) under influence of the presence of saponin during transfection of the nucleic acid into the cell in vitro, represents a novel property of saponins, which—to the knowledge of the inventors—was not existing and apparent so far. Cells, susceptible to saponin-based in vitro transfections (e.g. Neuro-2A, JIMT-1) can now be transfected with any nucleic acid, regardless their size, due to the current contribution by the inventors. Of importance, the saponin-based transfection of plasmid DNA with a plasmid size of larger than 5 kbp, e.g. even larger than 7-8 kbp, reaches a higher efficiency when compared to one of the methods currently used in the field, using transfection agent Lipofectamin3000. The problem the field is suffering from when desired transfection of relatively large plasmid DNA of e.g. 5.5 kbp or higher is considered, is for example illustrated by the following quote, taken from the ThermoFisher website in December 2020: “Despite similar uptake efficiencies in cationic lipid-mediated transfection, nuclear delivery of large plasmids is compromised compared with small plasmid molecules. This effect is observed using equivalent mass or molar concentrations of different-sized constructs, suggesting that nuclear delivery of plasmids may be limited by the rate of intracellular transit and that small plasmids evade degradation by rapid transit through the cytoplasm, rather than through the saturation of cellular defenses (Lukacs, et al., 2000; McLenachan et al., 2007).”. Taken from: www.thermofisher.com/de/de/home/references/gibco-cell-culture-basics/transfection-basics/guidelines-for-plasmid-dna-transfection.html.

The technique of the current invention, wherein saponins are applied as transfection booster for large constructs such as plasmid DNA, provides a solution to the apparent hampered transfection efficiency currently seen in the field.

Saponins offer a variety of applications in the field of transfections. The conducted tests and displayed examples and tests executed previously proved, that this applies in in-vitro settings and experiments. With the successful complexation of large DNA-plasmids (plasmid DNA) into poly-lysine-based nanoplexes and previously conducted nanoplex formulations with smaller sized plasmid-DNA (with a plasmid DNA size of up to 4-5 kbp), mRNA and mini-circle-DNA, the transfection triad consisting of nanoparticles composed of poly-lysine K₁₆ peptide and nucleic acid, combined with the presence of saponin when the nanoparticles are contacted with the cells that are to be transfected with the nucleic acid, now turned out to be an universal tool for efficient and non-toxic in vitro gene delivery, according to the invention.

With poly-lysine K₁₆ peptide-based nanoplexes, surprisingly DNA-plasmids of as large as 8.2 kbp could be complexed and transfected into cells in vitro, in the presence of (1-5 microgram/mL) GE1741 or SO1861. Based on the performed analyses, it was apparent that also larger-size poly-lysine peptides could form stable nanoplexes (gel retention assay). This provides flexibility with regard to the amount of positively charged peptides, which can be adjusted.

### REFERENCES

Clochard, J., Jerz, G., Schmieder, P., Mitdank, H., Troger, M., Sama, S., Weng, A., (2020) A new acetylated triterpene saponin from Agrostemma githago L. modulates gene delivery efficiently and shows a high cellular tolerance. Int J Pharm 589, 119822.
Ryu, N., Kim, M. A., Park, D., Lee, B., Kim, Y. R., Kim, K. H., Baek, J. I., Kim, W. J., Lee, K. Y., Kim, U. K., (2018) Effective PEI-mediated delivery of CRISPR-Cas9 complex for targeted gene therapy. Nanomedicine 14, 2095-2102.
Sama, S., Jerz, G., Schmieder, P., Joseph, J. F., Melzig, M. F., Weng, A., (2018a) Plant derived triterpenes from Gypsophila elegans M.Bieb. enable non-toxic delivery of gene loaded nanoplexes. J Biotechnol 284, 131-139.
Sama, S., G. Jerz, P. Schmieder, E. Woith, M. F. Melzig, and A. Weng. 2017. ‘Sapofectosid—Ensuring non-toxic and effective DNA and RNA delivery’, Int J Pharm, 534: 195-205.
Sama, S., E. Woith, W. Walther, G. Jerz, W. Chen, S. Hart, M. F. Melzig, and A. Weng. 2018b. ‘Targeted suicide gene transfections reveal promising results in nu/nu mice with aggressive neuroblastoma’, J Control Release, 275: 208-16.

SEQUENCES-SEQ ID NOS

SEQ ID NO: 1
Cas9 from Streptococcus pyogenes serotype M1;
sequence retrieved from www.uniprot.org/uniprot/Q99ZW2
>sp|Q99ZW2|CAS9_STRP1 CRISPR-associated endonuclease Cas9/Csn1
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLWVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
SEQ ID NO: 2
Cas12a from Francisella tularensis subsp. novicida (strain U112);
sequence retrieved from www.uniprot.org/uniprot/A0Q7Q2
>sp|A0Q7Q2|CS12A_FRATN CRISPR-associated endonuclease Cas12a
MSIYQEFVNKYSLSKTLRFELIPQGKTLENIKARGLILDDEKRAKDYKKAKQIIDKYHQF
FIEEILSSVCISEDLLQNYSDVYFKLKKSDDDNLQKDFKSAKDTIKKQISEYIKDSEKFK
NLFNQNLIDAKKGQESDLILWLKQSKDNGIELFKANSDITDIDEALEIIKSFKGWTTYFK
GFHENRKNVYSSNDIPTSIIYRIVDDNLPKFLENKAKYESLKDKAPEAINYEQIKKDLAE
ELTFDIDYKTSEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVTTMQSFYEQIA
AFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILA
NFAAIPMIFDEIAQNKDNLAQISIKYQNQGKKDLLQASAEDDVKAIKDLLDQTNNLLHKL
KIFHISQSEDKANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKF
IDFYKQSISKHPEWKDFGFRFSDTQRYNSIDEFYREVENQGYKLTFENISESYIDSVVNQ
GKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMKTNYHDKLAAI
EKDRDSARKDWKKINNIKEMKEGYLSQVVHEIAKLVIEYNAIVVFEDLNFGFKRGRFKVE
KQVYQKLEKMLIEKLNYLVFKDNEFDKTGGVLRAYQLTAPFETFKKMGKQTGIIYYVPAG
FTSKICPVTGFVNQLYPKYESVSKSQEFFSKFDKICYNLDKGYFEFSFDYKNFGDKAAKG
KWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANGAY
HIGLKGLMLLGRIKNNQEGKKLNLVIKNEEYFEFVQNRNN
SEQ ID NO: 3
Cas13a from Leptotrichia buccalis (strain ATCC 14201/DSM 1135/
JCM 12969/NCTC 10249/C-1013-b); sequence retrieved from
www.uniprot.org/uniprot/C7NBY4
>sp|C7NBY4|CS13A_LEPBD CRISPR-associated endoribonuclease Cas13a
MKVTKVGGISHKKYTSEGRLVKSESEENRTDERLSALLNMRLDMYIKNPSSTETKENQKR
IGKLKKFFSNKMVYLKDNTLSLKNGKKENIDREYSETDILESDVRDKKNFAVLKKIYLNE
NVNSEELEVFRNDIKKKLNKINSLKYSFEKNKANYQKINENNIEKVEGKSKRNIIYDYYR
ESAKRDAYVSNVKEAFDKLYKEEDIAKLVLEIENLTKLEKYKIREFYHEIIGRKNDKENF
AKIIYEEIQNVNNMKELIEKVPDMSELKKSQVFYKYYLDKEELNDKNIKYAFCHFVEIEM
SQLLKNYVYKRLSNISNDKIKRIFEYQNLKKLIENKLLNKLDTYVRNCGKYNYYLQDGEI
ATSDFIARNRQNEAFLRNIIGVSSVAYFSLRNILETENENDITGRMRGKTVKNNKGEEKY
VSGEVDKIYNENKKNEVKENLKMFYSYDFNMDNKNEIEDFFANIDEAISSIRHGIVHFNL
ELEGKDIFAFKNIAPSEISKKMFQNEINEKKLKLKIFRQLNSANVFRYLEKYKILNYLKR
TRFEFVNKNIPFVPSFTKLYSRIDDLKNSLGIYWKTPKTNDDNKTKEIIDAQIYLLKNIY
YGEFLNYFMSNNGNFFEISKEIIELNKNDKRNLKTGFYKLQKFEDIQEKIPKEYLANIQS
LYMINAGNQDEEEKDTYIDFIQKIFLKGFMTYLANNGRLSLIYIGSDEETNTSLAEKKQE
FDKFLKKYEQNNNIKIPYEINEFLREIKLGNILKYTERLNMFYLILKLLNHKELTNLKGS
LEKYQSANKEEAFSDQLELINLLNLDNNRVTEDFELEADEIGKFLDFNGNKVKDNKELKK
FDTNKIYFDGENIIKHRAFYNIKKYGMLNLLEKIADKAGYKISIEELKKYSNKKNEIEKN
HKMQENLHRKYARPRKDEKFTDEDYESYKQAIENIEEYTHLKNKVEFNELNLLQGLLLRI
LHRLVGYTSIWERDLRFRLKGEFPENQYIEEIFNFENKKNVKYKGGQIVEKYIKFYKELH
QNDEVKINKYSSANIKVLKQEKKDLYIRNYIAHFNYIPHAEISLLEVLENLRKLLSYDRK
LKNAVMKSVVDILKEYGFVATFKIGADKKIGIQTLESEKIVHLKNLKKKKLMTDRNSEEL
CKLVKIMFEYKMEEKKSEN

1. Use of a saponin in the in vitro delivery of a nucleic acid into a cell, wherein the saponin is a bisdesmosidic triterpenoid 12,13-dehydrooleanane-type saponin and wherein the nucleic acid comprises more than 5.5 kilo base-pairs (kbp).

2. Use according to claim 1, wherein the saponin is according to formula (I): 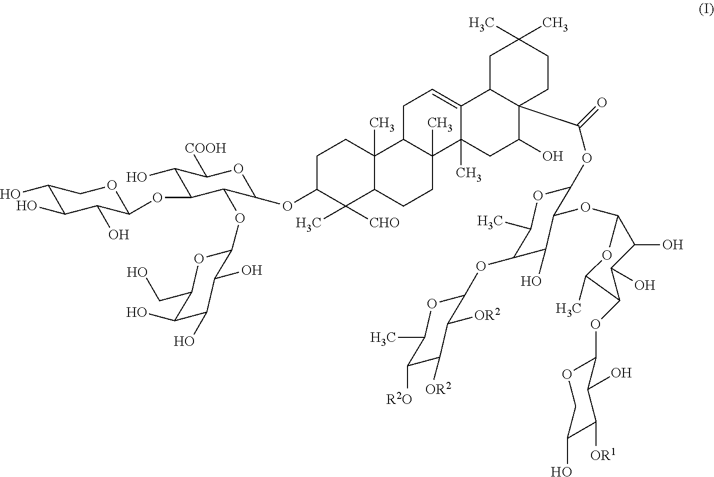
wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin,
preferably wherein R¹ of the saponin according to formula (I) is a xylose residue and/or in that the saponin carries exactly two acetyl groups,
more preferably wherein the acetyl groups of the saponin according to formula (I) are bonded to the oxygen atoms in C3 position and in C4 position of the corresponding quinovose residue of the saponin, or wherein one of the R² residues of the saponin according to formula (I) is a xylose residue, one of the R² residues is an acetyl group and one of the R² residues is H, preferably wherein the xylose residue of the saponin according to formula (I) is bonded to the oxygen atom in C3 position of the corresponding quinovose residue and in that the acetyl group is bonded to the oxygen atom in C4 position of the corresponding quinovose residue of the saponin,
or more preferably wherein R¹ of the saponin according to formula (I) is a xylose residue and in that two R² groups of the saponin according to formula (I) are acetyl groups which are bonded to the oxygen atoms in C3 position and in C4 position of the corresponding quinovose residue, and in that the third R² group is H.

3. Use according to claim 2, characterized in that the saponin is GE1741 according to formula (II): 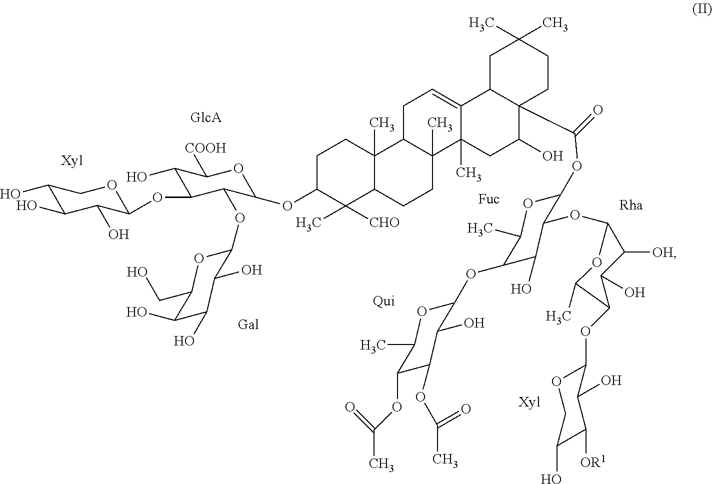
wherein R¹ is xylose;
or in that the saponin is SO1861 according to formula (111):

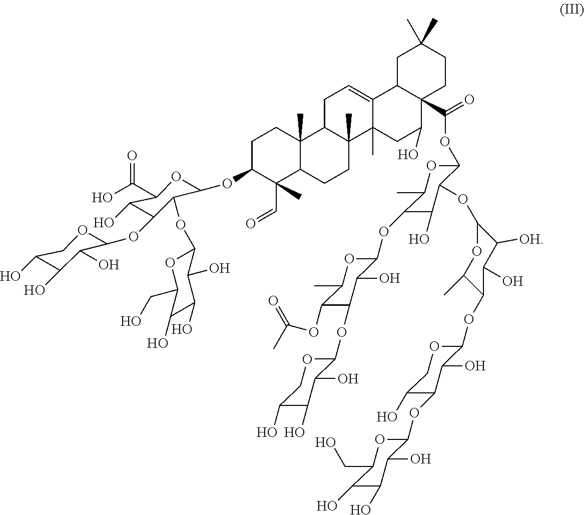

4. Use according to any one of the claims 1-3, characterized in that the nucleic acid is a plasmid DNA.

5. Use according to any one of the claims 1-4, characterized in that the nucleic acid forms part of a nanoparticle which nanoparticle further comprises a nanoparticle-forming compound, preferably wherein the nanoparticle-forming compound comprises or consists of a poly-lysine peptide, preferably wherein the poly-lysine peptide consists of 5-25 lysine residues, more preferably the poly-lysine peptide consists of 16 lysine residues.

6. Use according to claim 5, characterized in that the nanoparticle comprises the nanoparticle-forming compound and the nucleic acid in a mass ratio in a range of 3:1 to 15:1, preferably in a range of 3:1 to 9:1.

7. Use according to any one of the claims 1-6, characterized in that the nucleic acid at least comprises a first part of the nucleic acid that encodes for a CRISPR-associated endonuclease (Cas) and wherein the nucleic acid optionally comprises a second part of the nucleic acid that encodes for a guide RNA (gRNA) or is a gRNA or comprises a gRNA, preferably wherein the nucleic acid is a Cas-encoding plasmid DNA, more preferably in that the nucleic acid is a plasmid DNA for expressing at least the Cas, even more preferably in that the nucleic acid is a plasmid DNA for expressing the Cas and for expressing the gRNA.

8. Use according to any one of the claims 1-7, characterized in that the nucleic acid, preferably the plasmid DNA, more preferably the Cas-encoding plasmid DNA comprises more than at least 6 kbp, preferably at least 7 kbp, more preferably at least 7.5 kbp, most preferably at least 8 kbp.

9. Use according to any one of the claims 7-8, characterized in that the Cas encoding nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b).

10. Use according to any one of the claims 1-9, characterized in that the cell is a eukaryotic cell.

11. Method for delivering a nucleic acid comprising more than 5.5 kilo base-pairs (kbp) into a cell in vitro, comprising the steps of:
(i) providing the nucleic acid; and
(ii) providing a saponin that is a bisdesmosidic triterpenoid 12,13-dehydrooleanane-type saponin; and
(iii) incubating the cell with the nucleic acid in the presence of the saponin.

12. Method according to claim 11, wherein the saponin is according to formula (I): 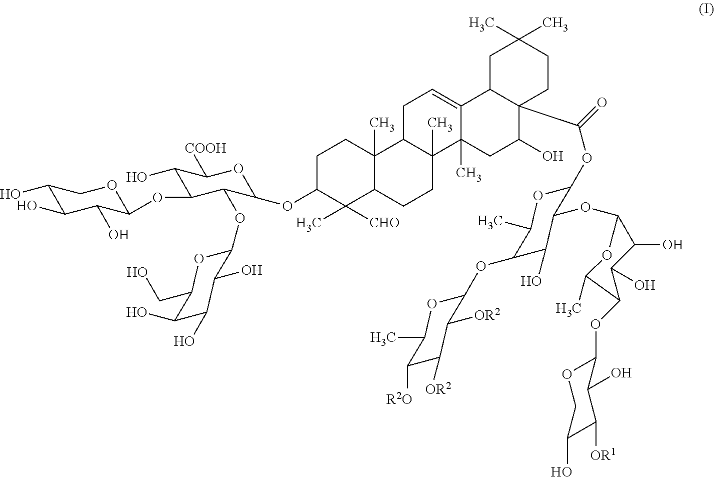
wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin.

13. Method according to claim 11 or 12, characterized in that the nucleic acid is a plasmid DNA.

14. Method according to any one of the claims 11 to 13, characterized in that the nucleic acid forms part of a nanoparticle which nanoparticle further comprises a nanoparticle-forming compound, preferably wherein the nanoparticle comprises the nanoparticle-forming compound poly-lysine peptide, wherein preferably the poly-lysine peptide consists of 5-25 lysine residues, more preferably the poly-lysine peptide consists of 16 lysine residues.

15. Method according to claim 13 or claim 14, characterized in that the nanoparticle comprises the nanoparticle-forming compound and the nucleic acid in a mass ratio in a range of 3:1 to 15:1, preferably in a range of 3:1 to 9:1.

16. Method according to any one of the claims 11 to 15, characterized in that the saponin concentration is 1 μg/mL-5 μg/mL in step (ii) of the method.

17. Method according to any one of the claims 11-16, characterized in that the saponin is GE1741 according to formula (II): 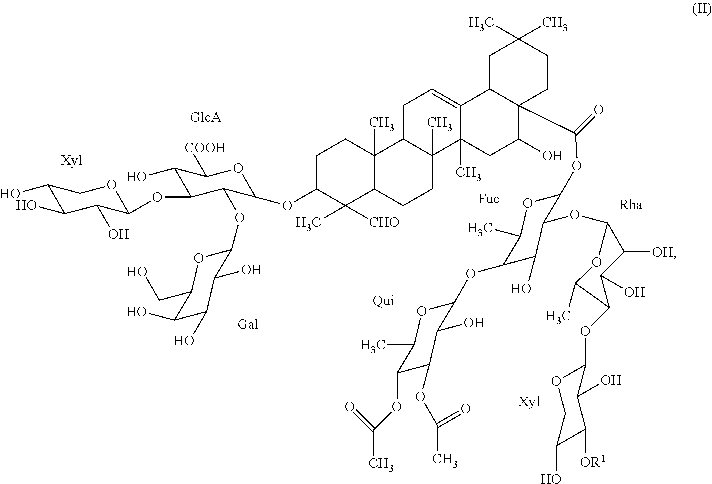
wherein R¹ is xylose;
or in that the saponin is SO1861 according to formula (III):

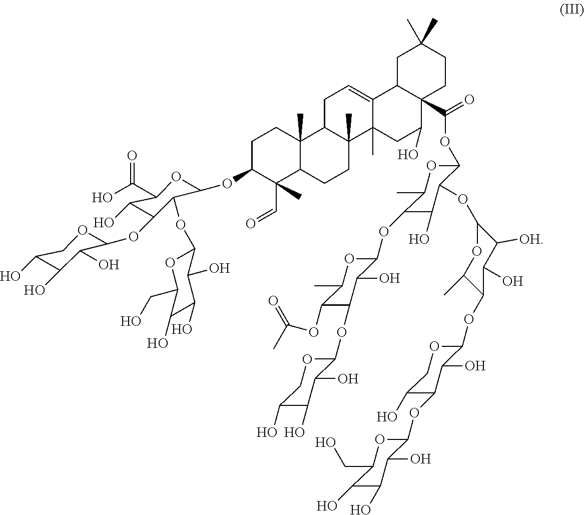

18. Method according to any one of the claims 12-17, characterized in that the nucleic acid is a nucleic acid encoding for the CRISPR/Cas construct that at least comprises a first part that encodes for a Cas and wherein the nucleic acid optionally comprises a second part that encodes for a gRNA or comprises a gRNA or is a gRNA, preferably wherein the nucleic acid encoding for the CRISPR/Cas construct is provided as part of a plasmid DNA, preferably in that at least the part of the nucleic acid encoding for the Cas part of the CRISPR/Cas construct is provided as part of a plasmid DNA, more preferably in that the plasmid DNA is for expressing the Cas and the gRNA of the CRISPR/Cas construct.

19. Method according to any one of the claims 12-18, characterized in that the nucleic acid, preferably the plasmid DNA comprises more than at least 6 kbp, preferably at least 7 kbp, more preferably at least 7.5 kbp, most preferably at least 8 kbp.

20. Method according to any one of the claims 11-19, characterized in that the Cas encoding nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b).

21. Kit of parts for delivering a nucleic acid comprising more than 5.5 kilo base-pairs (kbp) into a cell in vitro comprising:
a first combination comprising or consisting of:
(a) a first container comprising a first plasmid DNA;
(b) optionally a second container comprising a second plasmid DNA;
(c) a third container comprising poly-lysine K₁₆ peptide;
(d) a fourth container comprising at least one saponin being a bisdesmosidic triterpenoid 12,13-dehydrooleanane-type saponin, preferably being one saponin, most preferably selected of: GE1741 and SO1861;
(e) instructions for use,
wherein the use at least comprises the preparation of nanoplexes of the poly-lysine K₁₆ peptide and the first plasmid DNA, and optionally the saponin,
wherein the first plasmid has a size of at least 5.5 kbp,
or
a second combination comprising or consisting of:
(a) a first container comprising a third plasmid DNA;
(b) a second container comprising poly-lysine K₁₆ peptide;
(c) a third container comprising at least one saponin being a bisdesmosidic triterpenoid 12,13-dehydrooleanane-type saponin, preferably being one saponin, most preferably selected of: GE1741 and SO1861;
(d) instructions for use,
wherein the use at least comprises the preparation of nanoplexes of the poly-lysine K₁₆ peptide and the third plasmid DNA, and optionally the saponin,
wherein the third plasmid has a size of at least 5.5 kbp,
or
a third combination comprising or consisting of:
(a) a first container comprising the first plasmid DNA;
(b) optionally a second container comprising a RNA oligonucleotide;
(c) a third container comprising poly-lysine K₁₆ peptide;
(d) a fourth container comprising at least one saponin saponin being a bisdesmosidic triterpenoid 12,13-dehydrooleanane-type saponin, preferably being one saponin, most preferably selected of: GE1741 and SO1861;
(e) instructions for use,
wherein the use at least comprises the preparation of nanoplexes of the poly-lysine K₁₆ peptide and the first plasmid DNA, and optionally the saponin,
wherein the first plasmid has a size of at least 5.5 kbp,
preferably wherein the first plasmid DNA is encoding for Cas9, and the second plasmid DNA is encoding for gRNA and/or the RNA oligonucleotide is a gRNA RNA oligonucleotide, or wherein the third plasmid DNA is encoding for Cas9 and encoding for gRNA.

22. Composition comprising a nanoparticle, suitable for in vitro delivery of a nucleic acid into a cell, wherein the nanoparticle comprises or consists of:
(i) the nucleic acid comprising more than 5.5 kilo base-pairs (kbp);
(ii) a poly-lysine peptide, and
(iii) a saponin being a bisdesmosidic triterpenoid 12,13-dehydrooleanane-type saponin.

23. The Composition of claim 22, wherein the saponin is according to formula (I): 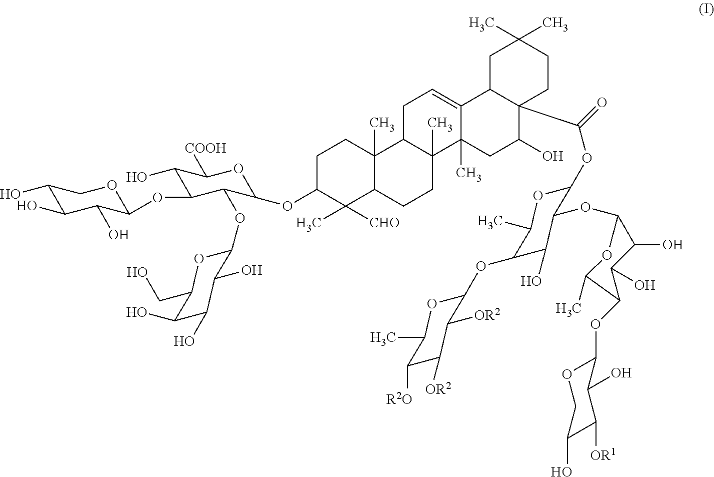
wherein
R¹ is a xylose residue, an arabinose residue, or a glucose residue bonded with its C1 atom to the corresponding xylose residue of formula (I); and
R² is independent from other R² residues in the same molecule H, an acetyl residue or a xylose residue bonded with its C1 atom to the corresponding quinovose residue of formula (I), with the proviso that at least two acetyl residues or at least one acetyl residue and a xylose residue bonded to the quinovose residue are present in the saponin,
preferably wherein R¹ of the saponin according to formula (I) is a xylose residue and/or in that the saponin carries exactly two acetyl groups
or wherein the acetyl groups of the saponin according to formula (I) are bonded to the oxygen atoms in C3 position and in C4 position of the corresponding quinovose residue of the saponin-more preferably wherein one of the R² residues of the saponin according to formula (I) is a xylose residue, one of the R² residues is an acetyl group and one of the R² residues is H,
even more preferably wherein the xylose residue of the saponin according to formula (I) is bonded to the oxygen atom in C3 position of the corresponding quinovose residue and in that the acetyl group is bonded to the oxygen atom in C4 position of the corresponding quinovose residue of the saponin, most preferably wherein R¹ of the saponin according to formula (I) is a xylose residue and in that two R² groups of the saponin according to formula (I) are acetyl groups which are bonded to the oxygen atoms in C3 position and in C4 position of the corresponding quinovose residue, and in that the third R² group is H.

24. The Composition according to claim 23, wherein the saponin is GE1741 according to formula (II): 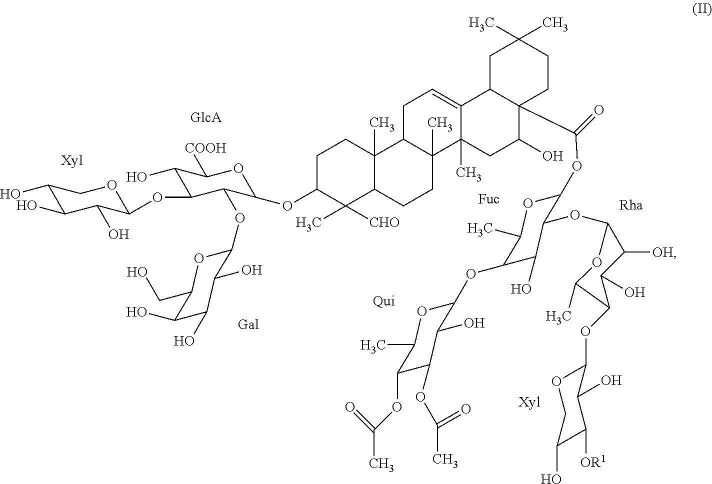
wherein R¹ is xylose;
or wherein the saponin is SO1861 according to formula (111):

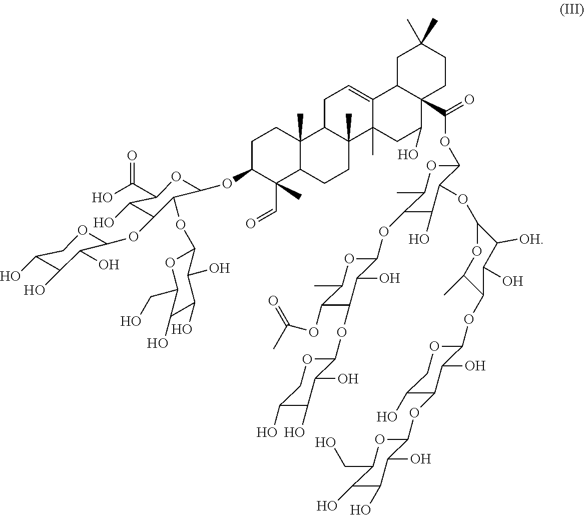

25. The Composition according to any one of the claims 22-24, wherein the poly-lysine peptide consists of 5-25 lysine residues, more preferably the poly-lysine peptide consists of 16 lysine residues.

26. The Composition according to any one of the claims 22-25, wherein the nanoparticle comprises the poly-lysine peptide and the nucleic acid in a mass ratio in a range of 3:1 to 15:1, preferably in a range of 3:1 to 9:1.

27. The Composition according to any one of the claims 22-26, wherein the nucleic acid is part of a plasmid DNA.

28. The Composition according to any one of the claims 22-27, wherein the nucleic acid at least comprises a first part of the nucleic acid that encodes for a Cas and wherein the nucleic acid optionally comprises a second part of the nucleic acid that encodes for a gRNA or comprises a gRNA or is a gRNA.

29. The Composition according to any one of the claims 22-28, wherein the nucleic acid, preferably the plasmid DNA comprises more than at least 6 kbp, preferably at least 7 kbp,-more preferably at least 7.5 kbp, most preferably at least 8 kbp.

30. The Composition according to any one of the claims 22-29, wherein the Cas encoding first part of the nucleic acid is encoding for Cas9 (SEQ ID NO: 1), Cas12a (SEQ ID NO: 2), Cas13a (SEQ ID NO:3) or a Cas with at least 90% amino-acid residue identity with any one of Cas9 of Streptococcus pyogenes serotype M1, Cas12a of Francisella tularensis subsp. novicida (strain U112) and Cas13a of Leptotrichia buccalis (strain ATCC 14201/DSM 1135/JCM 12969/NCTC 10249/C-1013-b).


SEQUENCE LISTING (3 sequences)

SEQ ID NO: 1 - protein, 1368 aa, Streptococcus pyogenes [sequence omitted]

SEQ ID NO: 2 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1159 aa, Leptotrichia bucallis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN